(19) United States
(12) Patent Application Publication
(10) Pub. No.: US 2026/0162805 A1
(43) Pub. Date: Jun. 11, 2026

(54) MULTI-MODAL RETRIEVAL AUGMENTED GENERATION FOR INTERACTIONS WITH DIGITAL VIDEOS

(71) Applicant: Intuitive Surgical Operations, Inc., Sunnyvale, CA (US)

(72) Inventors: Conor Perreault, Atlanta, GA (US); Kiran Bhattacharyya, Atlanta, GA (US); Anthony M. Jarc, Belmont, CA (US); Hong Seo Lim, Sunnyvale, CA (US); Ziheng Wang, Atlanta, GA (US); Aneeq Zia, Alpharetta, GA (US)

(73) Assignee: Intuitive Surgical Operations, Inc., Sunnyvale, CA (US)

(21) Appl. No.: 19/413,447

(22) Filed: Dec. 9, 2025

Related U.S. Application Data
(60) Provisional application No. 63/730,262, filed on Dec. 10, 2024.

(51) Int. Cl. G16H 30/20; A61B 34/00; A61B 34/10; G06V 20/40; G16H 30/40; G16H 50/70

(52) U.S. Cl. CPC G16H 30/20; A61B 34/10; A61B 34/25; G06V 20/41; G16H 30/40; G16H 50/70

(57) ABSTRACT

The technical solutions are directed to a multi-modal retrieval augmented generation for natural language interactions with surgical video and data. A system can include a processor coupled with memory. The processor can identify, for a medical procedure performed via a robotic medical system, a video stream and a plurality of data streams related to the medical procedure. The processor can determine, using one or more models trained with machine learning, based on the plurality of data streams, performance data for a clip of the video stream. The processor can transform the performance data and the clip to an embedding vector for an embedding space stored in a data repository. The processor can update the embedding space to provide, in response to a search query executed on the embedding space, access to at least a portion of the video stream of the medical procedure.

### CROSS-REFERENCES TO RELATED APPLICATIONS

This application claims benefit and priority under 35 U.S.C. § 119 to U.S. Provisional Patent Application No. 63/730262, filed Dec. 10, 2024, which is hereby incorporated by reference herein in its entirety.

### BACKGROUND

Medical procedures can be performed in an operating room with a robotic medical system. As the amount and variety of equipment in the operating room increases, or medical procedures become increasingly complex, it can be challenging for robotic medical systems to perform such medical procedures efficiently, reliably, or without incident.

### SUMMARY

The technical solutions of this disclosure establish relationships between multi-modal data, such as surgical videos and robotic data, to provide contextual search engine based multi-modal user interaction. Review and analysis of surgical videos and robotic surgical data can be complex and time consuming, resulting in time consuming and compute resource and energy inefficient system performance. As data for analysis of new techniques can include various performance metrics corresponding to different measures for determining medical procedure outcomes, it can be challenging to timely and efficiently identify the related information across different data modalities. As a result, analysis of such data becomes even more difficult. The technical solutions of this disclosure can overcome these challenges by creating semantic mappings between data modalities in the context of multi-modal data streams of recorded medical procedures to provide quick as well as compute and energy efficient searchable user interactions with the system across the multi-modal data using natural language inputs.

The technical solutions introduced herein provide a performance metrics-driven machine learning (ML) based user guidance platform to improve surgical outcomes for robotic system medical procedures. In the course of ongoing medical procedures, robotic medical systems can gather various procedure related data, such as different types of data streams and performance metrics on various stages of the medical procedure, reflecting on opportunities for systematic improvement of the surgical outcome. However, the lack of real-time system-based insights into such opportunities can undermine their timely and intraoperative identification. This can impact the surgical success of the medical procedure as it can be challenging to maximize the likelihood of a desired surgical outcome given the absence of a solution to identify and notify the surgeon of such opportunities, which can arise in a variety of situations.

Thus, the data processing system described herein can address technical challenges as well as challenges faced by practitioners and researchers working with complex surgical and medical data. By providing a unified, intuitive platform for searching, analyzing, and contextualizing multi-modal data (e.g., video, sensor, and performance metrics), the data processing system described herein can streamline workflows, reduces manual effort, and provide new insights that were previously challenging or not possible obtain efficiently, reliably or accurately, resulting in benefits for end users in clinical (e.g., pre-operatively, post-operatively, or even intra-operatively), research, and educational settings.

An aspect of the technical solutions is directed to a system. The system can include one or more processors that are coupled with memory. The one or more processors can be configured (e.g., via instructions or data stored in the memory) to identify, for a medical procedure performed via a robotic medical system, a video stream and a plurality of data streams related to the medical procedure. The one or more processors can be configured to determine, using one or more models trained with machine learning, based on the plurality of data streams, performance data for a clip of the video stream. The one or more processors can be configured to transform the performance data and the clip to an embedding vector for an embedding space stored in a data repository. The one or more processors can be configured to update the embedding space to provide, in response to a search query executed on the embedding space, access to at least a portion of the video stream of the medical procedure.

The one or more processors can be configured to receive, from the robotic medical system, the plurality of data streams comprising at least one of a kinematics data stream, an event stream, or a non-robotic data stream. The one or more models can comprise a generative artificial intelligence model. The one or more processors can be configured to generate, using the generative artificial intelligence model, the performance data based on the plurality of data streams. The performance data can include a text-based description of the clip generated from the plurality of data streams.

The one or more processors can be configured to generate, using the one or more models, a plurality of performance metrics based on the plurality of data streams. The one or more processors can be configured to generate, using generative artificial intelligence, the performance data based on the plurality of performance metrics. The one or more processors can be configured to provide a graphical user interface for a search engine and receive, via the graphical user interface, the search query. The one or more processors can be configured to select, based on execution of the search query on the embedding space, a search result corresponding to the medical procedure and provide the search result for display via the graphical user interface.

The one or more processors can be configured to execute the search query using a distance-based nearest neighbor search. The one or more processors can be configured to execute the search query using a linear model. The one or more processors can be configured to execute the search query via interpolation through a generative embedding space to identify the search result, wherein the search result comprises synthetic data.

The one or more processors can be configured to display, via a graphical user interface, the clip of the medical procedure. The one or more processors can be configured to receive, during the display of the clip, via the graphical user interface, a query related to the medical procedure. The one or more processors can be configured to execute the query on the embedding space to select the performance data associated with the clip and provide a response to the query based at least in part on the performance data.

The one or more processors can be configured to update the embedding space with a plurality of embedding vectors constructed for a plurality of clips of the video stream. The one or more processors can be configured to aggregate performance data for at least two of the plurality of clips. The one or more processors can be configured to generate an aggregated embedding vector for the aggregated performance data. The one or more processors can be configured to update the embedding space with the aggregated embedding vector. The one or more processors can be configured to update the embedding space with a plurality of embedding vectors constructed for a plurality of clips of a plurality of video streams of a plurality of medical procedures.

An aspect of the technical solutions are directed to a method. The method can include one or more processors coupled with memory identifying, for a medical procedure performed via a robotic medical system, a video stream and a plurality of data streams related to the medical procedure. The method can include determining, by the one or more processors, using one or more models trained with machine learning, based on the plurality of data streams, performance data for a clip of the video stream. The method can include transforming, by the one or more processors, the performance data and the clip to an embedding vector for an embedding space stored in a data repository. The method can include updating, by the one or more processors, the embedding space to provide, in response to a search query executed on the embedding space, access to at least a portion of the video stream of the medical procedure.

The method can include the one or more processors receiving, from the robotic medical system, the plurality of data streams comprising at least one of a kinematics data stream, an event stream, or a non-robotic data stream. The one or more models can comprise a generative artificial intelligence model. The method can include generating, by the one or more processors, using the generative artificial intelligence model, the performance data based on the plurality of data streams. The performance data can include a text-based description of the clip generated from the plurality of data streams.

The method can include generating, by the one or more processors, using the one or more models, a plurality of performance metrics based on the plurality of data streams. The method can include generating, by the one or more processors, using generative artificial intelligence, the performance data based on the plurality of performance metrics. The method can include the one or more processors providing a graphical user interface for a search engine. The method can include the one or more processors receiving, via the graphical user interface, the search query. The method can include selecting, by the one or more processors, based on execution of the search query on the embedding space, a search result corresponding to the medical procedure. The method can include providing, by the one or more processors, the search result for display via the graphical user interface.

The method can include the one or more processors executing the search query using a distance-based nearest neighbor search. The method can include executing, by the one or more processors, the search query using a linear model. The method can include executing, by the one or more processors, the search query via interpolation through a generative embedding space to identify the search result, wherein the search result comprises synthetic data.

The method can include the one or more processors displaying, via a graphical user interface, the clip of the medical procedure. The method can include receiving, by the one or more processors, during the display of the clip, via the graphical user interface, a query related to the medical procedure. The method can include executing, by the one or more processors, the query on the embedding space to select the performance data associated with the clip. The method can include providing, by the one or more processors, a response to the query based at least in part on the performance data.

The method can include updating, by the one or more processors, the embedding space with a plurality of embedding vectors constructed for a plurality of clips of the video stream. The method can include aggregating, by the one or more processors, performance data for at least two of the plurality of clips. The method can include generating, by the one or more processors, an aggregated embedding vector for the aggregated performance data. The method can include updating, by the one or more processors, the embedding space with the aggregated embedding vector. The method can include the one or more processors updating, the embedding space with a plurality of embedding vectors constructed for a plurality of clips of a plurality of video streams of a plurality of medical procedures.

An aspect of the technical solutions is directed to a non-transitory computer-readable medium storing processor executable instructions. The instructions, when executed by one or more processors, can cause the one or more processors to identify, for a medical procedure performed via a robotic medical system, a video stream and a plurality of data streams related to the medical procedure. The instructions, when executed by one or more processors, can cause the one or more processors to determine, using one or more models trained with machine learning, based on the plurality of data streams, performance data for a clip of the video stream. The instructions, when executed by one or more processors, can cause the one or more processors to transform the performance data and the clip to an embedding vector for an embedding space stored in a data repository. The instructions, when executed by one or more processors, can cause the one or more processors to update the embedding space to provide, in response to a search query executed on the embedding space, access to at least a portion of the video stream of the medical procedure.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIG. 1 depicts an example system for multi-modal retrieval augmented generation for natural language interactions with surgical video and data.

FIG. 2 illustrates an example of a surgical system, in accordance with some aspects of the technical solutions.

FIG. 3 illustrates an example block diagram of an example computer system is shown, in accordance with some aspects of the technical solutions.

FIG. 4. illustrates an example configuration with an embedding space implemented using a multi-modal vector database providing relationships between different modes of data.

FIG. 5 illustrates an example configuration for generating embedding vectors from data streams of the robotic medical system.

FIG. 6 illustrates an example flow diagram of a method for a multi-modal retriever augmented generation for natural language reactions.

### DETAILED DESCRIPTION

Following below are more detailed descriptions of various concepts related to, and implementations of, systems, methods, apparatuses for multi-modal retrieval augmented generation for natural language interactions with surgical video and data. The various concepts introduced above and discussed in greater detail below can be implemented in any of numerous ways.

Although the present disclosure is discussed in the context of a surgical procedure, in various aspects, the technical solutions of this disclosure can be applicable to other medical or non-medical applications, treatments, sessions, environments or activities, in which performance metrics based user guidance for robotic systems can be sought. For instance, technical solutions can be applied in any environment, application or industry in which activities, operations, processes or acts by robots or robotic tools involve performance metrics that can be used to provide a platform for user guidance while utilizing robotic systems.

The technical solutions of this disclosure establish relationships between multi-modal data, including medical procedure videos and robotic data, to provide for contextual search engine-based multi-modal user interaction. The review and analysis of surgical videos and robotic surgical data present significant challenges due to their inherent complexity and time-consuming nature. Moreover, the performance metrics corresponding to various measures of medical procedure outcomes can vary widely across different medical procedures, patients, or data modalities, making their collection very difficult. As a result, efficient identification and extraction of the relevant multi-modal data for a given medical procedure or its performance metrics can be very challenging, as well as compute resource and energy inefficient, making any analysis of such multi-modal data that much more difficult.

The technical solutions of this disclosure overcome these challenges by utilizing machine learning techniques to create an embedding space defining relationships between different portions of data across different data modalities, such as video clips as well as sensor, kinematics or events robotic data. The technical solutions can transform performance data and video clips into embedding vectors allowing for more effective comparisons and searches. An embedding vector can refer to or include, for example, a numerical representation of a portion of data, such as a video clip, sensor reading, or text description. The embedding vector can be generated using machine learning models to capture the key features and contextual relationships of the portion of data. Embedding vectors allow for efficient comparison and semantic search across different data modalities by mapping them into a shared embedding space.

In doing so, the technical solutions described herein allow for execution of natural language based search queries to retrieve the relevant video clips based on semantic mappings established between the different data modalities. As a result, the technical solutions facilitate quick and accurate processing of search queries, providing efficient search results across multiple data types in a computationally and energy-efficient manner.

The technical solutions described herein provide specific, practical improvements to computer technology by providing efficient, real-time, and semantically meaningful retrieval and analysis of multi-modal medical data using advanced machine learning models. For example, the data processing system described herein can improve performance relative to systems that use keyword or metadata searches. To do so, the data processing system described herein can use embedding vectors and a unified multi-modal vector database to establish semantic relationships between diverse data types (e.g., video, sensor, kinematics, and text), thereby allowing for context-aware search and retrieval based on the generation of embedding vectors from multi-modal data, the use of generative artificial intelligence models to produce performance data and synthetic data, and the implementation of a unified embedding space for semantic search, resulting in improved accuracy, speed, and utility in the analysis of complex medical procedures.

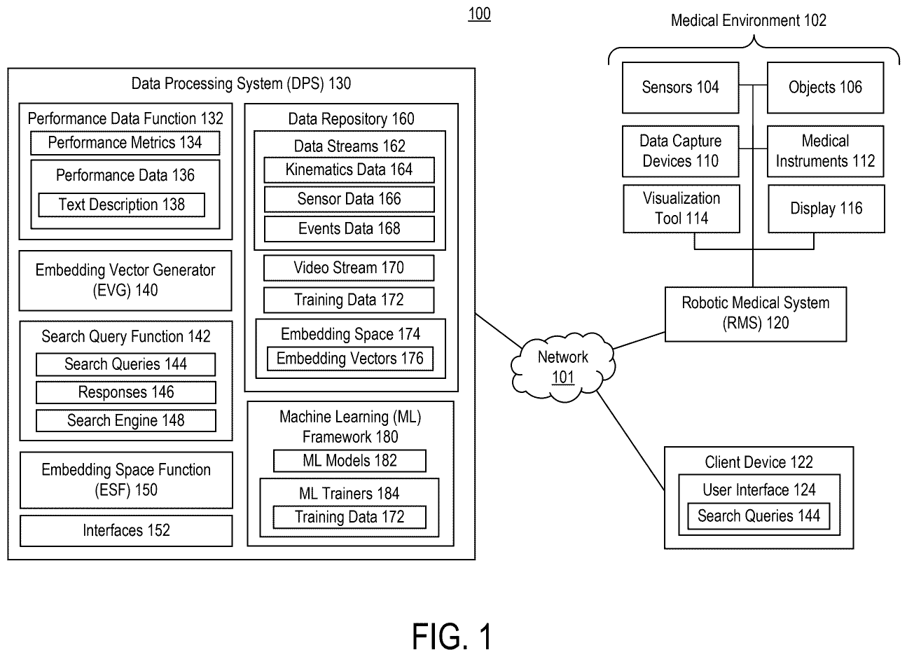

FIG. 1 depicts an example system 100 for multi-modal retrieval augmented generation for natural language interactions with surgical video and data. The system 100 can include a medical environment 102 (e.g., a medical facility or a surgical room) that can include one or more of sensors 104, objects 106, data capture devices 110, medical instruments 112, visualization tools 114 and displays 116. The medical environment 102 can include one or more of robotic medical system (RMS) 120 configured to facilitate, perform or be used during performance of medical procedures, such as robotic surgeries. The RMS 120 can be communicatively coupled, via network 101, with one or more of client devices 122 and data processing systems 130.

Client device 122 can include a computing device (e.g., a computing system, a laptop, a tablet or a smartphone) for a client or a user to use for execution or utilization of an application interfacing with the data processing system 130. The application can include operate one or more user interfaces 124 which a user of the client device 122 can utilize to generate search queries 144. The search queries 144 can include textual descriptions or request seeking explanations or answers related to various details of a medical procedure, referring for example to any phase, tasks or actions taken by a surgeon in the course of the procedure performance.

Data processing system 130 can include a combination of hardware and software for providing a multi-modal retrieval augmented generation for natural language interactions with surgical video and data. Data processing system 130 can include one or more of performance data functions 132 for determining performance data 136 and embedding vector generators (EVGs) 140 for transforming performance data 136 into embedding vectors 176. Data processing system 130 can include one or more of search query functions 142 for processing search queries 144 from client devices 122 and embedding space functions (ESFs) 150 for generating, adjusting or updating the embedding space 174 with its embedding vectors 176 to provide access to particular video streams 170 or data streams 162 responsive to the search queries 144. Data processing system 130 can include one or more of interfaces 152 for interfacing with and exchanging communications with the user interfaces 124 of the client devices 122, as well as data repositories 160 for storing various data and one or more machine learning (ML) frameworks 180 for providing ML various functionalities. A performance data function 132 can generate or use one or more performance metrics 134 and identify, generate or determine one or more performance data 136, which can include text description 138. A search query function 142 can receive and execute one or more search queries 144 and use one or more search engines 148 to generate responses 146 for the given queries 144. Data repository 160 can store and provide access to video stream 170 and various data streams 162, such as streams of kinematics data 164, sensor data 166 or events data 168. Data repository can store and provide access to training data 172 for ML trainers 184 and embedding space 174 that can include any number of embedding vectors 176. ML framework 180 can include one or more ML models 182 trained by the ML trainers 184 using training data 172 to implement various functionalities of the data processing system components (e.g., performance data function 132, EVG 140, search query function 142 and ESF 150).

System 100 can a robotic medical system 120, also referred to as RMS 120, which can include any medical robot (e.g., surgical robot) that is configured for performing medical tasks or procedures, such as by using medical instruments. The RMS 120 can include robotic arms for holding and maneuvering surgical instruments, one or more high-definition 3D cameras for providing views of the surgical site, and one or more consoles for allowing a user (e.g., a surgeon) to operate or maneuver the arms and tools of the RMS to perform surgeries. The robotic arms of the RMS 120 can be configured to translate movements of the user on the console or a user interface of the RMS into smaller and more accurately controlled movements of medical instruments 112 while performing the medical procedure (e.g., a medical surgery on a patient).

The medical environment 102 can include any arrangement of sensors 104, objects 106, data capture devices 110, medical instruments 112, visualization tools 114 and displays 116 utilized with the RMS 120 to perform a medical procedure. The objects 106 can include any type of objects or articles, such as medical operating tables, shelves, holders, various medical instruments separate from those used by the RMS 120, surgical lights, medical equipment carts, imaging equipment or other systems or tools for carrying fluids or patient monitoring equipment. The data capture devices 110 (e.g., optical devices, such as image or video cameras, as well as microphones, radio frequency identification (RFID) readers, data loggers, smartphone or tablet devices or depth sensors) can be used for logging or capturing any data streams 162. The data streams 162 can include any sequence or stream of data, including sequence or stream of any sensor data 166 (e.g., data from sound sensors, video cameras or other sensors), events data 168 (e.g., data on logs of events or occurrences involving an RMS 120) and kinematics data 164 (e.g., movements of medical instruments 112). The visualization tools 114 to gather the captured data streams 162 and process it for display to the user (e.g., a surgeon or other medical professional) at one or more displays 116, including any tool for 3D representation of a medical environment 102. The visualization tool 114 can include a system for processing data and generating visualizations (e.g., simulations or illustrations) using a display 116. The display 116 can present data stream 162 (e.g., video frames, data on events, kinematics or sensor readings) of an ongoing medical procedure (e.g., an ongoing surgery) performed using the RMS 120 as it handles, manipulates, holds or otherwise utilizes medical instruments 112 to perform surgical tasks at the surgical site.

Data capture devices 110 can include any of a variety of sensors, cameras, video imaging devices, infrared imaging devices, visible light imaging devices, intensity imaging devices (e.g., black, color, grayscale imaging devices, etc.), depth imaging devices (e.g., stereoscopic imaging devices, time-of-flight imaging devices, etc.), medical imaging devices such as endoscopic imaging devices, ultrasound imaging devices, etc., non-visible light imaging devices, any combination or sub-combination of the above mentioned imaging devices, or any other type of imaging devices that can be suitable for the purposes described herein. Data capture devices 110 can include cameras that a surgeon can use to perform a surgery and observe manipulation components within a purview of field of view suitable for the given task performance.

Data capture devices 110 can capture, detect, or acquire sensor data, such as videos or images, including for example, still images, video images, vector images, bitmap images, other types of images, or combinations thereof. The data capture devices 110 can capture the images at any suitable predetermined capture rate or frequency. Settings, such as zoom settings or resolution, of each of the data capture devices 110 can vary as desired to capture suitable images from any viewpoint. For instance, data capture devices 110 can have fixed viewpoints, locations, positions, or orientations. The data capture devices 110 can be portable, or otherwise configured to change orientation or telescope in various directions. The data capture devices 110 can be part of a multi-sensor architecture including multiple sensors, with each sensor being configured to detect, measure, or otherwise capture a particular parameter (e.g., sound, images, or pressure).

Data capture devices 110 can include any type and form of a sensor 104 that can be configured to measure and provide sensor data 166, including a positioning sensor, a biometric sensor, a velocity sensor, an acceleration sensor, a vibration sensor, a motion sensor, a pressure sensor, a light sensor, a distance sensor, a current sensor, a focus sensor, a temperature sensor, a haptic or tactile sensor or any other type and form of sensor used for providing data on medical tools 112, or data capture devices (e.g., optical devices). Sensor 104 can include a depth sensor configured to determine a distance between the sensor and an object (e.g., distance to a medical instrument 112 or a patient's anatomy). For example, a data capture device 110 can include a location sensor, a distance sensor or a positioning sensor providing coordinate locations of a medical tool 112 or a data capture device 110. Data capture device 110 can include a sensor providing information or data on a location, position or spatial orientation of an object (e.g., medical tool 112 or a lens of data capture device 110) with respect to a reference point. The reference point can include any fixed, defined location used as the starting point for measuring distances and positions in a specific direction, serving as the origin from which all other points or locations can be determined.

Display 116 can show, illustrate or play data streams 162, including video data, in which medical tools 112 at or near surgical sites are shown. For example, display 116 can display a rectangular image (e.g., a frame of a video data) of a surgical site along with at least a portion of medical instruments 112 being used to perform surgical tasks. Display 116 can provide compiled or composite images generated by the visualization tool 114 from a plurality of data capture devices 110 to provide visual feedback from one or more points of view.

Visualization tool 114 that can be configured or designed to receive any number of different data streams 162 from any number of data capture devices 110 and combine them into a single data stream displayed on a display 116. The visualization tool 114 can be configured to receive a plurality of data stream components and combine the plurality of data stream components into a single data stream 162. For instance, the visualization tool 114 can receive a visual sensor data from one or more medical tools 112, sensors or cameras with respect to a surgical site or an area in which a surgery is performed. The visualization tool 114 can incorporate, combine or utilize multiple types of data (e.g., positioning data of a medical tool 112 along sensor readings of pressure, temperature, vibration or any other data) to generate an output to present on a display 116. Visualization tool 114 can combine or correlate various data streams 162 based on their respective time of generation, using for example, metadata indicative of time of each portion of data stream 162 (e.g., timestamps in the metadata) to match the data across the data streams 162 to use for determinations.

Medical instruments or tools 112 can be any type and form of tool or instrument used for surgery, medical procedures or a tool in an operating room or environment. Medical tool 112 can be imaged by, associated with or include an image capture device and can be handled or maneuvered using robotic manipulator arms 235 of the RMS 120. For instance, a medical tool 112 can be a tool for making incisions, a tool for suturing a wound, an endoscope for visualizing organs or tissues, an imaging device, a needle and a thread for stitching a wound, a surgical scalpel, forceps, scissors, retractors, graspers, or any other tool or instrument to be used during a surgery. Medical tools 112 can include hemostats, trocars, surgical drills, suction devices or any instruments for use during a surgery. The medical tool 112 can include other or additional types of therapeutic or diagnostic medical imaging implements. The medical tool 112 can be configured to be installed in, coupled with, or manipulated by an RMS 120, such as by manipulator arms 235 or other components for holding, using and manipulating the medical instruments 112 during procedure.

RMS 120 can be a computer-assisted system configured to perform a surgical or medical procedure or activity on a patient via or using or with the assistance of one or more robotic components or medical tools 112. The RMS 120 can be deployed in any medical environment 102, such as any space or facility for performing medical procedures (e.g., surgical procedures), including for example any surgical facility or an operating room. The medical environment 102 can include medical instruments 112, which the RMS 120 can use for performing various actions or tasks of a medical procedure, including any invasive, non-invasive, in-patient, or out-patient tasks or procedures. RMS 120 can include configurations that can provide or include various settings, configurations, adjustments, operating parameters or constraints for controlling movements, motion or actions performed using the RMS 120. RMS 120 can include any number of manipulator arms (e.g., 235) for grasping, holding or manipulating various medical tools 112 and performing computer-assisted medical tasks using medical tools 112 controlled by the manipulator arms.

Client device 122 can include any combination of hardware and software for facilitating creation and sending of search queries 144 to a data processing system 130. Client device 122 can include a computer (e.g., a workstation or a laptop), a tablet or a smartphone. Client device 122 can execute or operate one or more applications for using functionalities of the data processing system 130 or for accessing data generated by the RMS 120. Client device 122 can be configured for network communication (e.g., via network 101, such as the internet or a WLAN network) with RMS 120, data processing system 130 or any components of the medical environment 102, including sensors 104, objects 106, data capture devices 110, medical instruments 112, visualization tools 114 and displays 116.

Client device 122 can include its own display 116 for displaying a user interface 124 that can facilitate a user with accessing and using the data processing system 130 functionalities. User interface 124 can include a graphical user interface (GUI) with any number of windows, menus, buttons or selection options for the user to enter or select search queries 144 and read the corresponding responses 146. The user interface 124 can include a search bar in a window that can include an autocomplete or suggestion functionalities to facilitate search query generation. User interface 124 can receive responses 146 from the data processing system 130, via interfaces 154 and present them for display via display 116 at the client device 122.

Search queries 144, also referred to as queries 144, can include any string of characters that can be used for generating responses 146 from the data processing system 130. Search queries 144 can be directed to medical procedures or surgical data science, covering any range of inquiries facilitating understanding of various components related to medical procedures. A search query 144 can seek information about a particular medical procedure actions of a task, tasks of a medical procedure phase or the medical procedure itself. A search query 144 can be directed to performance metrics 134, such as objective performance indicators (OPIs). For instance, a search query 144 can include a statement, such as “explain this OPI to me,” in order to gain insights into operational performance indicators or request clarifications on surgical techniques.

Search query 144 can include or describe multimedia elements, such as description of a video clip for a particular action (e.g., movement of a scalpel) for a particular task including a sequence of actions (e.g., incision) or a plurality tasks of a medical procedure. Search query 144 can include a statement, such as “take me to the sections where the surgeon fires a stapler in the prior surgery,” linking their inquiry to a precise moment in a video stream 170 of a particular medical procedure. The search queries 144 can be comparative, as in “how does the performance in this surgery compare to other performances in the existing literature?” enabling users to contextualize particular data against established research.

Responsive to sending a search query 144, the client device 122 can receive from the data processing system 130, a response 146 generated responsive to the search query 144. The response 146 can include any output (e.g., an answer) to a query 144 generated by a search query function 142. Responses 146 can be generated by the search query 144 using one or more ML models 182 trained to provide responses 146, such as responses including portions of the video stream 170 or data stream 162 corresponding to the search query 144. The search query function 142 can generate the responses 146 responsive to queries 144 input into one or more ML models 182 trained to perform embedding processes. The ML models 182 can be trained to generate a vector representation for one or more portions of the search query 144. The search query function 142 can provide responses 146 for transmission to the client device 122 via an interface 152. Depending on configuration, responses 146 can be output in various forms, including text, audio, or visual feedback, depending on the nature of the interaction. Responses 146 can include, for example, one sentence answers, paragraphs of description, portions of documents, any one or more portions of video stream 170 (e.g., video clip) or any corresponding data stream portion (e.g., kinematics data 164, sensor data 166 or events data 168 corresponding to the video clip).

Network traffic, such as search queries 144 and responses 146, can be communicated between the data processing system 130 and the client devices 122 via one or more networks 101. A network 101 can include any type or form of a communication network. The geographical scope of the network 101 can vary widely and can include a body area network (BAN), a personal area network (PAN), a local-area network (LAN) (e.g., Intranet), a metropolitan area network (MAN), a wide area network (WAN), or the Internet. The topology of the network 101 can assume any form such as point-to-point, bus, star, ring, mesh, tree, etc. The network 101 can utilize different techniques and layers or stacks of protocols, including, for example, the Ethernet protocol, the internet protocol suite (TCP/IP), the ATM (Asynchronous Transfer Mode) technique, the SONET (Synchronous Optical Networking) protocol, the SDH (Synchronous Digital Hierarchy) protocol, etc. The TCP/IP internet protocol suite can include application layer, transport layer, internet layer (including, e.g., IPv6), or the link layer. The network 101 can be a type of a broadcast network, a telecommunications network, a data communication network, a computer network, a Bluetooth network, or other types of wired and wireless networks.

Data processing system 130 can include any combination of hardware and software for providing a multi-modal retrieval of data based on natural language interactions or requests. The data processing system 130 can be deployed in or associated with the medical environment 102, or it can be provided on a server or a cloud-based function that is remote from the medical environment 102. The data processing system 130 can include one or more interfaces 152 designed, constructed and operational to communicate data (e.g., exchange search queries 144 and responses 146), with one or more client devices 122, or with RMS 120 (e.g., data streams 162 or video streams 170), via network 101. Data processing system 130 can be implemented using instructions stored in memory locations and processed by one or more processors, controllers or integrated circuitry. For instance, data processing system 130 components or functionalities (e.g., performance data function 132, EVG 140, search query function 142, ESF 150 or interface 152) can be implemented using instructions, commands or data stored on memory 315 and accessed and executed by one or more processors 310.

The data processing system 130, as well as any of its components or functionalities can be served or provided in using any one or more technologies. For instance, data processing system 130 or its components can a part of or include a cloud computing environment functionality or features or include a group of logically grouped servers implemented via various distributed computing techniques. The logical group of servers may be referred to as a data center, server farm or a machine farm. The servers can be centered within data center or geographically dispersed. A data center or machine farm may be administered as a single entity, or the machine farm can include a plurality of machine farms. The servers within each machine farm can be heterogeneous—one or more of the servers or machines can operate according to one or more type of operating system platform.

The data processing system 130, or components thereof, can be located at least partially at the location of the surgical facility associated with the medical environment 102 or remotely therefrom. Elements of the data processing system 130, or components thereof can be accessible via portable client devices 122, such as laptops, mobile devices, wearable smart devices, etc. The data processing system 130, or components thereof, can include other or additional elements that can be considered desirable to have in performing the functions described herein. The data processing system 130, or components thereof, can include, or be associated with, one or more components or functionality of a computing including, for example, one or more processors 310 coupled with memory (e.g., 315 or 325) that can store instructions, data or commands for implementing the functionalities of the DPS 130 discussed herein.

Data repository 160 of the DPS 130 can be any combination of hardware and software for storing and providing access to data. Data repository 160 can include a hard drive or a cloud storage, such as for example, a storage device 325. Data repository 160 can include one or more data streams 162 of various types and from various sources, including measurements from sensors 104, which can be referred to as sensor data 166. Sensor data 166 can include data from video cameras (e.g., images or video frames), or various force, torque or biometric data, haptic feedback data, pressure or temperature data, vibration, tension or compression data, endoscopic images or data, ultrasound images or videos or communication and command data streams. Data repository 160 can include events data 168, such as data on medical instrument 112 or other component installation, uninstallation, configuration, reconfiguration, setting or resetting data or information related to system files or logs. ML models 182 or their functionalities (e.g., ML framework 180 components) can each be partially or fully stored in a data repository 160, along with training data sets (e.g., 178) and data streams 162. Data repository can store data streams 162, video streams 170 and training data 172. Data repository 160 can store and provide access to (e.g., per request or application programming interface or API call) embedding space 174 which can include a plurality of embedding vectors 176 organized in a multi-model database or data structure.

The data repository 160 can include one or more data files, data structures, arrays, values, or other information that facilitates operation of the data processing system 130, such as a database for a multi-modal embedding space 174. The data repository 160 can include one or more local or distributed databases and can include a database management system. The data repository 160 can include, maintain, or manage a data stream 162. The data stream 162 can include or be formed from one or more of a video stream, image stream, stream of sensor measurements, event stream, or kinematics stream. The data stream 162 can include data collected by one or more data capture devices 110, such as a set of 3D sensors from a variety of angles or vantage points with respect to the procedure activity (e.g., point or area of surgery).

Data stream 162 can include a stream of kinematics data 164, which can refer to or include data associated with one or more of the manipulator arms or medical tools 112 (e.g., instruments) attached to the manipulator arms, such as arm movements, locations or positioning. Data corresponding to medical tools 112 can be captured or detected by one or more displacement transducers, orientational sensors, positional sensors, or other types of sensors and devices to measure parameters or generate kinematics information. The kinematics data 164 can include sensor data along with time stamps and an indication of the medical tool 112 or type of medical tool 112 associated with the data stream 162.

Video stream 170 can include any stream or sequence of media, including images or video frames or clips. Video stream 170 can include video data, such as images or videos captured by a medical tool 112 (e.g., endoscopic camera) can be sent to the visualization tool 114. The robotic medical system 120 can include one or more input ports to receive video stream 170 via direct or indirect connection of one or more auxiliary devices. For example, the visualization tool 114 can be connected to the RMS 120 to receive the images from the medical instrument 112 when the medical instrument 112 is installed in the RMS 120 (e.g., on a manipulator arm of the RMS 120 that is used for moving, managing or otherwise handing medical instruments 112). The visualization tool 114 can combine the data streams 162 from the data capture devices 110 and the medical tool 112 into a single combined data stream 162 for use by the ML framework 180.

Embedding space 174 can include any type and form of framework that maps different data types or modes, such as text, images, and sensor readings, into a vector space to capture the relationships and similarities between such data across different types or modes of data. Embedding space 174 can include or represent embedding vectors 176 generated from multiple modes of data, such as video streams 170, where each frame or clip is represented by an embedding vector that captures visual features and contextual information. Embedding space 174 can include embedding vectors of kinematic data 164 from a robotic medical system 120, reflecting the movement patterns and trajectories of medical instruments 112 during medical procedures. Embedding space 174 can represent specific sensor data 166, such as temperature or pressure readings from the robotic medical system 120, or particular events data 168 (e.g., time of installation or engagement of a medical instrument 112). Embedding space 174 can define or represent various modes of data (e.g., video, sensor, kinematics, or events) along with the relationships or correlations between them using embedding vectors 176. Embedding space 174 can facilitate real-time search query implementation and identification of relevant data based on comparisons between the vector representation of a search query and the embedding vectors 176 within the embedding space 174. In doing so, the embedding space 174 can allow for efficient identification of one or more chunks of data to provide with the response 146 to a search query 144, across different modes of data. For example, a response 146 for a search query 144 can identify embedding vectors 176 for a portion of a video stream 170 (e.g., a video clip of a task), which can be provided along with a corresponding set of sensor data 166 (e.g., sensor readings contemporaneous with the video clip), or corresponding kinematics data 164 (e.g., information on force or direction of medical instrument movements contemporaneous with the video clip).

Embedding space 174 can facilitate analysis of data by the various data types or modes being integrated into a unified framework, allowing for richer insights and more nuanced search queries 144. For instance, embedding vectors 176 can represent temporal sequences in video streams, where each vector captures not only visual features but also the dynamics of actions over time, such as “Show me the sequence of events during suturing.” For instance, embedding space 174 can include embedding vectors 176 derived from audio data, capturing sound features during surgical procedures, which can be correlated (e.g., via matching timestamps in the metadata also represented in the embedding vectors 176) with visual and kinematic data to provide a comprehensive understanding of the surgical environment. For example, embedding vectors 176 can represent patient-specific data, such as demographics or medical history, allowing for personalized analysis and improved decision-making. By establishing relationships between these diverse embedding vectors 176 across modalities—video, audio, kinematics, sensor data, and patient information—embedding space 174 can enhance search functionalities. For example, a search query like “find similar procedures based on this patient's data” can leverage the embedding space 174 to retrieve relevant video clips and corresponding sensor readings that match the specified criteria.

Embedding vectors 176, also referred to as vectors 176, can be any numerical representations of data (e.g., portion of video stream 170 or data stream 162) that can correspond to points within an embedding space 174. Embedding vectors 176 can be generated by embedding vector generator 140, including for example EVG 140 utilizing one or more ML models 182 trained to generate embedding vectors 176. Embedding vectors 176 can include numerical representations (e.g. collection of values organized as a vector) indicative of specific features of the portion of data to which they correspond or any relationships of that data (e.g., a video clip within a video stream 170) to other data modalities (e.g., portion of data stream 162 corresponding to sensor data 166 captured during the same time interval as the time interval of the video clip).

Each embedding vector 176 can correspond to or encode specific attributes of the data it represents, such as visual characteristics in video frames, movement dynamics in kinematic data from an RMS 120, or environmental conditions in sensor readings. For example, an embedding vector 176 derived from a video stream 170 can include any visual elements of a surgical procedure it captures (e.g., a scalpel making an incision on a specific tissue). For example, an embedding vector 176 derived from the video stream 170 can capture contextual information, such as lighting conditions or medical instrument positioning. For example, embedding vectors 176 representing kinematic data 164 can indicate the force, the speed or trajectory of medical instruments 112, providing insights into their operational efficiency that can be combined with the information from the corresponding video clip capturing the same time interval. For instance, embedding vectors 176 can be generated from events data, capturing relevant occurrences, such as tool engagements and disengagements, which can inform the workflow dynamics or help more correctly identify the relevant portions of the video stream 170. By maintaining relationships between these diverse embedding vectors 176, including within the same data modality and across different data modalities (e.g., between different types of data), the data processing system 130 can facilitate providing accurate responses 146 for complex search queries 144. For instance, a search query like “Compare instrument movements during different procedures” can utilize the corresponding embedding vectors 176 to identify and analyze similarities and differences in kinematic patterns across various surgical video streams 170 or data streams 162 (e.g., sensor data 168).

Performance data function 132 can include any combination of hardware and software for identifying, determining or generating performance data 136. Performance data function 132 can include the instructions, commands, executables files or data for identifying or selecting at least a portion of video streams 170 or data streams 162 related to a medical procedure for which to determine or generate performance data 136. The identification or selection of the portions of video or data streams can be implemented using one or more ML models 182 trained to select multi-modal (e.g., multiple types of) data, including video data, kinematics data, sensor data or events data. The performance data 136 can be identified or generated for a portion of a video stream (e.g., a video clip) of a medical procedure performed via a robotic medical system 120, such as a robotic surgery. The performance data function 132 can determine or generate the performance data 136 based on data streams 162 (e.g., sequences of sensor, kinematics, events or video data).

Performance data function 132 can receive and identify data streams 162 corresponding to one or more robotic medical procedures implemented using one or more RMSs 120. The performance data function 132 can utilize an ML model 182 that is a generative artificial intelligence (GenAI) model to generate or produce the performance data, such text description 138. A GenAI model can refer to or include, for example, a machine learning model that is configured to generate new content, such as text, images, or data, by learning patterns and structures from existing datasets. GenAI models can generate performance data, including text-based descriptions, from multi-modal surgical data streams, thereby allowing for advanced search and analysis functionalities. The text description 138 of the performance data 136 can include a text-based description of the clip. The text-based description of the clip can be generated based on the plurality of data streams 162 (e.g., data streams for various types of sensor, events, kinematics or other data). The data streams 162 can be input into the Gen AI model or be used for training the Gen AI model 182. For example, the data streams 162 can be used by the embedding vector generator 140 to generate embedding vectors 176 that can be used to generate or produce training data 172 for training the ML model 182 (e.g., GenAI model).

The performance data function 132 can generate performance metrics 136. The performance metrics 136 can include scores, rankings or values indicative of performance of a user (e.g., surgeon) performing any individual action or a phase of medical procedure. For instance, a performance metric 134 can correspond to a ranking or percentage rating of performance a surgeon performing the portion of medical procedure with respect to a dataset of all performance metrics 136 of all surgeons performing the same portion of the medical procedure. The performance metrics can be based on any combination of data streams 162, including streams of sensor data 166, kinematics data 164 or events data 168. The performance data function 132 can generate the performance data 136 based on any one or more performance metrics 134 generated using the data streams 162.

Performance metrics 134 (e.g., OPIs) can include any values, indicators or metrics for any aspect or portion of medical procedures performed using RMS 120, such as any medical procedure, its various phases or actions for each of the phases. Performance metrics 134 can include any text description 138 of performance, such as values, indicators or metrics indicative of a surgeon's ability to perform particular aspects of a medical procedure. Performance metrics 134 or text descriptions 138 of the performance data, can include values indicative of aspects of surgeon's productivity, quality of care, timeliness, or specialized skills. Performance metrics 134 level of consistency with which particular tasks related to one or more medical procedures, patients or surgeons. Performance metrics 134 can be indicative of a surgeon's productivity, quality, timeliness, customer satisfaction, specialized skills or abilities, success rates with respect to particular medical procedures, their phases within the medical procedure, tasks within any phases or actions within any task.

Performance metrics 134 can include any type and form of OPIs. For example, performance metric 134 can include an OPI of a duration, which can be expressed in the units of minutes and can correspond to a total time spent to perform a particular case, phase or a step. Performance metric 134 can include an OPI of a maximum force, which can be expressed in the units of Newtons (N) and correspond to the maximum detected force for a medical instrument. Performance metric 134 can include an OPI of an average force, which can be expressed in N and correspond to the average detected force for a medical instrument. Performance metric 134 can include an OPI of a time above a threshold N of force (e.g., time above 6.5N), which can be expressed in the units of % and correspond to the percentage of time with force applied above the threshold force amount. Performance metric 134 can include an OPI of an endoscope clutch count, which can be expressed in the units of numbers of a count, and which can correspond to the number of endoscope clutches performed on the console. Performance metric 134 can include an OPI of a hand controller clutch count, which can be expressed in numbers of a count, and which can correspond to the number of finger clutches performed on either hand controller on this console. Performance metric 134 can include an OPI of an energy pedal count, which can be expressed in a number of a count and correspond to the number of energy pedal presses initiated on the console.

Performance metric 134 can include an OPI of a total instrument path length, which can be expressed in meters and correspond to the path length traveled by all instrument tips on all manipulator arms of the RMS 120. Performance metric 134 can include an OPI of a total instrument angular path length, which can be expressed in radians and correspond to the total angular path length traveled by all instruments on all arms. Performance metric 134 can include an OPI of a hand controller movement percentage, which can be expressed in % and correspond to the proportion of time this hand controller was in motion, relative to the total time either hand controller was in motion on the given console. Performance metric 134 can include an OPI of a console movement percentage, which can be expressed in % and correspond to the proportion of time either hand controller was in motion on this console, relative to the total duration. Performance metric 134 can include an OPI of an instrument movement duration, which can be expressed in minutes and correspond to the total time the tip of the particular instrument type was in motion on any manipulator arm of the RMS 120. Performance metric 134 can include an OPI of a hand controller movement duration, which can be expressed in minutes and correspond to the total time this hand controller was in motion on this console. Performance metric 134 can include an OPI of a console movement duration, which can be expressed in minutes and correspond to the total time either hand controller was in motion on this console. Performance metric 134 can include an OPI of an arm swap count, which can be expressed in swaps and correspond to the number of arm swaps performed on the given console. Performance metric 134 can include an OPI of a head out count, which can be expressed in the number or count of events and which can correspond to the number of head out events on this console. Performance metric 134 can include an OPI of a head out rate, which can be expressed in a count over a time period (e.g., 1/hr) and which can correspond to the rate of head out events on this console.

Performance data 136 can be any information generated or identified with respect to a medical procedure. Performance data 136 can include qualitative or quantitative information, which can include, or be determined based on, various performance metrics 134. Performance data 136 can correspond to or include assessment of the effectiveness or efficiency of the medical procedures. Performance data 136 can include numerical performance metrics 134, such as the duration of specific surgical tasks, maximum and average forces applied by instruments, and counts of tool engagements. Performance data 136 can include a text description 138 that provides context or insights into the surgical process. For example, a text description 138 can summarize the key actions taken during a surgical clip, highlighting particular moments such as tool engagement or patient response.

Performance data 136 can be derived from multiple sources, including video streams 170, kinematic data 164 from robotic systems, sensor readings 166, and events data 168. Performance data 136 can combine, integrate or refer to these multi-modal data types allowing for a comprehensive evaluation of surgical performance across different data modes. For instance, performance data 136 can include or refer to performance metrics 134 that can indicate how a surgeon's actions compare to established benchmarks or best practices within a dataset of similar procedures. In cases where text descriptions 138 are included, the text descriptions 138 can improve the understanding by providing narrative context around the numerical metrics, such as detailing a surgeon's technique during a complex maneuver.

Embedding vector generator (EVG) 140 can include any combination of hardware and software for generating embedding vectors 176. EVG 140 can include the functionality (e.g., any combination of instructions, commands, executables, computer code or data) for transforming the performance data 136 or any portion of a video stream 170 (e.g., a video clip from the video stream) into embedding vectors 176, including embedding vectors 176 to include or integrate into an embedding space 174. EVG 140 can generate embedding vectors 176 from diverse data modalities, including any combination of sensor data 166, kinematics data 164, events data 168 or video data (e.g., video stream 170).

For instance, EVG 140 can generate embedding vectors 176 from audio signals captured during surgical procedures, which can be correlated with visual (e.g., video) and kinematic data using timestamps, which can be included in metadata of the multi-modal data (e.g., video or data stream portions) or their respective embedding vectors 176. For instance, EVG 140 can create embedding vectors 176 from real-time sensor readings, such as temperature, force, distance, depth or pressure readings (e.g., from sensors 104). Each generated embedding vector 176 can represent specific attributes, such as the speed and trajectory of a robotic arm 235 during surgery or the engagement status of surgical tools. EVG 140 can utilize ML techniques, such as machine learning models 182 trained for embedding tasks to generate the embedding vectors 176 across any modalities (e.g., data streams 162 or video stream 170), including any relations (e.g., correlations, contextual relation or time synchronization) between them.

Search query function 142, can include any combination of hardware and software for executing or processing search queries 144 and providing responses 146 to the search queries 144. Search query function 142 can include the functionality or an interface to provide a user on a client device 122 with an access to ML functionality. Search query function 142 can include ML-powered interface facilitating interaction between users and DPS 130 using LLM and NLP based ML models 182. For instance, search query function 142 can receive search queries 144, from a client device 122, via an interface 152. The search query 144 can include a textual description of a user question or request, which can correspond to a particular video stream 170 or data stream 162 portion (e.g., a video clip and its corresponding sensor, events or kinematics data). Search query function can utilize the EVG 140 to generate embedding vectors 176 corresponding to the search query 144, such as embedding vectors indicative of the contextual meaning of the type of data (e.g., video or data stream data) that the search query 144 is looking for.

Search query function 142 can include a parser function to parse and preprocess the textual input. Search query function 142 can process the text of the search query 144 using one or more selected ML models 182 suitable for a given query 144. ML models 182 can process the search query 144 within its context and provide response 146. For instance, search query function 142 can utilize ML models 182 to extract from the search query 144, a portion of the query 144 that can be input into one or more ML models 182 to perform the search or matching in the search engine 148. Search query function 142 can function as an intermediary, delivering these responses 146 back to the user, via a and allowing the user to enter new queries 144 for additional responses.

Search query function 142 can utilize the search engine 148 to generate or identify the response 146 for the search query 144. The response 146 can include a portion of a clip (e.g., portion of a video stream 170) whose embedding vector 176 was most similar or more closely corresponding to the embedding vector 176 of the search query 144. The response 146 can include other relevant modality (e.g., types) of data corresponding to the event or occurrence requested in the search query 144, such as sensor readings or kinematic information, which can be temporally aligned (e.g., cooccurring or occurring simultaneously) with the identified video segment of video clip. For example, if a user queries “Show me instances of wound suturing and the performance data for it”, the search engine 148 can return the video clip most relevant (e.g., most highly ranked cosine similarity search result for video clips) to the search query 144. The search engine 148 can also provide sensor data or kinematics data corresponding to (e.g., co-occurring with or occurring simultaneously with) the video clip. The search engine 148 can provide sensor or kinematics data whose embedding vectors 176 most closely correspond to (e.g., semantic search similarity) the embedding vectors 176 of the search query.

The search engine 148 can be any combination of hardware and software for retrieving and presenting information in response to search queries 144 input into the search engine 148. The search engine 148 can include or be coupled with the embedding space 174 such that the search query function 142 can utilize the search engine 148 to identify responses 146 (e.g., matching portions of video or data streams) to generate responses 146 (e.g., comprising the matching data) based on the embedding space 174 and its embedding vectors 176. The search engine 148 can index and catalogue various data modalities (e.g., data types), including video streams, sensor readings, and kinematic data, allowing for efficient retrieval based on embedding vectors 176. When a search query 144 is received, the search engine 148 can analyze analyzes or compare the query's embedding vector against indexed embedding vectors 176 within the embedding space 174 to identify matches. The matches can include the portions of the data streams 162 or portions of video stream 170 (e.g., video clips) whose embedding vectors 176 are contextually most similar to the embedding vectors 176 of the search query 144.

The search engine 148 or the search query function 142 can use any type of semantic searching process to execute the search query 144 and identify the data most closely corresponding to the search query 144 to use for the response 146. For instance, the search query function 142 can utilize approximate nearest neighbor (ANN) techniques, to quickly find and rank results based on their semantic similarity rather than mere keyword matches. For instance, if a user inputs a search query 144, such as “Find similar surgical techniques to the one just viewed,” the search engine can leverage its indexed data to return relevant video clips, along with associated performance metrics 134 and sensor data 166 that reflect similar procedural characteristics. For instance, the search query function 142 can execute the search query 144 using a distance-based nearest neighbor search. For instance, the search query function 142 execute the search query 144 using a linear model. For instance, the search query function 142 can execute the search query 144 via interpolation through a generative embedding space to identify the search result (e.g., the response 146). The search result (e.g., the response 146) can include, for example synthetic data.

Synthetic data can refer to or include, for example, data that is artificially generated by machine learning models, such as generative artificial intelligence, rather than being directly measured or recorded. Synthetic data can be produced by interpolating within a generative embedding space, allowing the system to simulate or represent scenarios not present in the original dataset and to enhance search and analysis capabilities. For instance, the EVG 140 can utilize ML models 182 to generate synthetic data, such as data generated based on moment parameters (e.g., parameters randomly generated based on one or more median or average vector values and a predetermined variance or standard deviation of a probability curve for the vector value).

For example, a user device 122 can display, via a graphical user interface 124, a clip of the medical procedure, such as a portion of a video stream 170 corresponding to a time interval (e.g., one or more seconds) of a video recording of a surgical procedure. The search query function 142 can receive, during the display of the clip, via the graphical user interface 124, a search query 144 related to the medical procedure. The search query 144 can request more information about the particular medical procedure, a particular phase of a medical procedure, a particular task or action within the phase of a medical procedure or a surgeon performing the medical procedure. The search query function 142 can execute the search query 144 on the embedding space 174 to select the performance data 136 associated with the clip. The performance data 136 can include a text description 138 of a given task, phase or medical procedure, or textual description or data (e.g., OPIs) of a surgeon implementing the medical procedure. The search query function 142 can generate and provide a response 146 to the search query 144 based at least in part on the performance data 136 (e.g., including any text description 138).

Embedding space function (ESF) 150 can include any combination of hardware and software for generating or updating the embedding space 174. ESF 150 can be generated or updated to correlate or create relations between the embedding vectors of the search queries 144 and the embedding vectors 176 of the portions of data streams 162 (e.g., 164, 166 or 168) or portions of video stream 170 (e.g., video clips or frames of medical procedure). ESF 150 can update the embedding space 174 to provide access to a portion of a video stream 170 of a medical procedure in response to a search query 144 being executed by the search query function 142 on the embedding space 174.

For instance, when a search query 144 is received requesting instances of tool engagement, ESF 150 can update the embedding space 174 to reflect new relationships between the embedding vectors 176 corresponding to both the search query 144 and relevant video segments that depict those engagements. For instance, when a new kinematic data is received updating the movement patterns of surgical instruments, ESF 150 can integrate these embedding vectors 176 into the existing embedding space 174. For instance, ESF 150 can facilitate continuous learning by adapting the embedding space 174 in real-time as new data streams 162 or video streams 170 are processed. For example, if a new surgical procedure is introduced and recorded, ESF 150 can incorporate embedding vectors 176 from this data into the embedding space 174, updating the embedding space 174. For instance, ESF 150 can analyze user interaction patterns with previous search queries 144 to refine how embedding vectors 176 are correlated. If certain types of search queries 144 consistently yield specific results, ESF 150 can adjust the relationships within the embedding space 174 to prioritize those results for similar future queries.

ESF 150 can be configured (e.g., via instructions in memory 315 for access and execution by processor 310) to update the embedding space with a plurality of embedding vectors 176 constructed for a plurality of clips of the video stream 170. ESF 150 can aggregate performance data 136 for at least two of the plurality of clips and generate an aggregated embedding vector 176 for the aggregated performance data 136 (e.g., for the at least two clips). The ESF 150 can update the embedding space 174 with the aggregated embedding vector 176. For instance, the ESF 150 can update the embedding space 174 with a plurality of embedding vectors 176 constructed for a plurality of clips of a plurality of video streams 170 of a plurality of medical procedures.

Machine learning (ML) framework 180 can include any combination of hardware and software for providing machine learning functionalities of the data processing system 130. ML framework 180 can include and utilize ML trainer 184 to use training data 172 to train one or more ML models 182. ML framework 180 can include various ML architecture or functions, such as attention mechanisms, large language models (LLMs), neural networks, transformers with encoder and decoder architecture, or any other type and form of ML architecture or functionality. ML framework 180 can be configured to facilitate effective determinations by the ML models 182 using, for example performance metrics 134 (e.g., OPIs) for various portions of medical procedures, including phases of a medical procedure, tasks of a phase or actions making up a task. ML framework 180 can include attention mechanisms which can utilize weights to improve the capacity of the ML models to discern, detect or recognize specific details within a context, improving the accuracy of determination, detection and prediction.

ML models 182 can be trained, configured or set up to implement or process any actions, recognitions, identifications, predictions, determinations or processing for, or on behalf of any functions of the data processing system 130. For instance, ML models 182 can be trained and configured to generate or identify performance data 136 or performance metrics 134. ML models 182 can be trained or configured to generate or modify (e.g., on behalf of EVG 140) embedding vectors 176 for various modalities of data (e.g., video streams 170 or data streams 162). ML models 182 can be trained or configured to perform searches (e.g., identify contextual similarities) for search queries 144 to generate responses 146 using search engine 148 or embedding space 174. The ML model 182 can be trained to generate or update the embedding space 174 (e.g., on behalf of the ESF 150).

The ML models 182 can include any generative AI models, which can include any machine learning systems configured to create new content, such as text, images, or audio, by learning patterns from the data, such as training data 172. ML models 182, which can also be sometimes include or be referred to as the generative AI models 182 or Gen AI models 182, can be trained using techniques, such as supervised learning, unsupervised learning, and reinforcement learning. Generative AI models 182 can utilize training data 172 to create logical inferences between various complex structures in the data set to generate coherent outputs.

The generative AI models 182 can include any machine learning (ML) or artificial intelligence (AI) model designed to generate content or new content, such as text, images, or code, by learning patterns and structures from existing data. The generative AI model 182 can be any model, a computational system or an algorithm that can learn patterns from data (e.g., chunks of data from various input documents, computer code, templates, forms, etc.) and make predictions or perform tasks without being explicitly programmed to perform such tasks. The generative AI model 182 can refer to or include a large language model. The generative AI model 182 can be trained using a dataset of documents (e.g., text, images, videos, audio or other data). The generative AI model 182 can be designed to understand and extract relevant information from the dataset. The generative AI model 182 can leverage natural language processing techniques and pattern recognition to comprehend the context, match it with relevant information in the training data, and generate a response that addresses the search query 144.

The generative AI model 182 can be built using deep learning techniques, such as neural networks, and can be trained on large amounts of data. The generative AI model 182 can be designed, constructed or include a transformer architecture with one or more of a self-attention mechanism (e.g., allowing the model to weigh the importance of different words or tokens in a sentence when encoding a word at a particular position), positional encoding, encoder and decoder (multiple layers containing multi-head self-attention mechanisms and feedforward neural networks). For example, each layer in the encoder and decoder can include a fully connected feed-forward network, applied independently to each position. The data processing system 130 can apply layer normalization to the output of the attention and feed-forward sub-layers to stabilize and improve the speed with which the generative AI model 182 is trained. The data processing system 130 can leverage any residual connections to facilitate preserving gradients during backpropagation, thereby aiding in the training of the deep networks. Transformer architecture can include, for example, a generative pre-trained transformer, a bidirectional encoder representations from transformers, transformer-XL (e.g., using recurrence to capture longer-term dependencies beyond a fixed-length context window), text-to-text transfer transformer,

The generative AI model 182 can be trained (e.g., by a model training function) using any text-based, video-based or data stream-based datasets by converting the text data from the input dataset documents into numerical representations (e.g., embeddings or embedding vectors 176) of the chunks of those documents, videos or data streams. These embeddings can capture the semantic meaning of words, paragraphs, pages, sensor readings or sentences, depending on the size and type of chunks of dataset documents are parsed into. Embeddings (e.g., embedding vectors 176) can be used to represent and organize the dataset documents within a high-dimensional space (e.g., embedding space), where similar documents, videos, sensor readings or concepts are located closer together. Embedding space can include a multi-dimensional vector space where each data point is represented by an embedding.

Through training, the generative AI model 182 can learn, or adjust its understanding of mapping the embeddings to particular issues (e.g., prompts related to resource availability or constraints concerning the resources), by adjusting its internal parameters. Internal parameters can include numerical values of the generative AI model 182 that the model learns and adjusts during training to optimize its performance and make more accurate predictions. Such training and can include iteratively presenting the various data chunks or documents of the dataset (e.g., or their chunks, embeddings) to the generative AI model 182, comparing its predictions with the known correct answers, and updating the model's parameters to minimize the prediction errors. By learning from the embeddings of the dataset data chunks, the generative AI model 182 can gain the ability to generalize its knowledge and make accurate predictions or provide relevant insights.

The generative AI model 182 can include any ML or AI model or a system that can learn from a dataset to generate new content (e.g., text or images) that resembles a distribution of the training dataset (e.g., synthetic data). A distribution of a dataset can include an underlying probability distribution representing the patterns and characteristics of the data used to train a generative AI model 182. For example, a training data distribution can represent statistical properties of a text data (e.g., text corpus), such as the frequency of words, the co-occurrence of terms, and the overall structure of the language used in the training dataset. The generative AI model 182 can include the functionality to utilize such a probability distribution of patterns and characteristics to generate new responses (e.g., predictions) that were not present in the dataset.

The ML trainer 184 can any combination of hardware and software for training ML models 182. Machine learning (ML) trainer 184 can include or generate ML models 182, each of which can be trained using training datasets that can include various data streams 162 corresponding to medical procedures using the RMS 120. ML trainer 184 can include a framework or functionality for training different types of ML models 182, such as LLMs, neural network models, spatial-temporal attention mechanism models or any other types of ML models 182. ML trainer 184 can include the functionality to utilize training data 172 for training ML models 182. ML trainer 184 can include the functionality for supervised or unsupervised learning or providing reinforcement learning algorithms for various types of ML models. ML trainer 184 can include the functionality for generating natural language processing, time series forecasting and recommendation ML systems.

Training data 172 can include any information or data used by the ML trainer 184 to train an ML model 182. Training data 172 can include documentation, RMS 120 records or logs, data streams 162, hospital records, performance metrics 134, or medical procedure actions, tasks or phases. Training data 172 can include information on various surgeons and their historical performance data, procedure characteristics of various patients, or any other information corresponding to medical procedures using RMS 120. Training data 172 can include one or more collections of medical documents, medical journal publications, research papers, surgical procedures, medical data from various medical procedures, each of which can be organized in ontologies, including tables that can interrelate various types of data for ML framework purposes.

Data processing system 130 can include an interface 152 to communicate with client devices 122. The interface 152 can include any type and form of an interface, including any combination of hardware and software, for communicating with the applications comprising user interfaces 124 on client devices 122. The interface 152 can include or operate an application, such as a web browser application or an application configured to execute on the data processing system 130 to receive search queries 144 from the client devices 122 and provide generated responses 146 (e.g., including any video clips (e.g., portions of videos stream 170) and related portions of data streams 162 (e.g., sensor, kinematics or events data).

The example system 100 can be deployed in a variety of products, services or scenarios. For instance, the example system 100 can be deployed in a service or product, such as an application for a surgical data science librarian. The application can be an application deployed on an interface 152 or a user interface 124 or a combination of the two. Such an application can allow users on the client device 122 to ask a data processing system 130 various natural language questions relating to surgical data science. The natural language used to generate search queries 144 can provide context to data that they are seeking. For example, when a user is presented with a set of performance indicators or metrics (e.g., OPIs) to describe a specific performance in a particular robotic surgical step, phase or a procedure, the application for the surgical data science librarian can be used to provide a description of the OPI and relevant research, allowing the user to ask any follow up questions. For instance, if a user is examining their own data and trying to determine how their results may fit into the broader context of surgical data science, the application can be used to help conduct a literature review and place new research in the context of existing research.

For example, system 100 can be utilized to provide an application (e.g., via interface 152 or user interface 124) to allow for surgical video question and answer services to client devices 122. For instance, this application can allow users on the client devices 122 to ask natural language or combined natural language +video questions. For instance, search queries 144 can describe what is happening in the video clips, providing textual descriptions of the portion of the video recording the user is seeking, which the data processing system 130 can utilize to generate responses 146 with the described video clip.

Such example search queries 144 can include, for instance, requests to show all the sections of a video where a user is using a particular medical instrument 122 (e.g., firing a stapler). For instance, a search query 144 can ask a question on what step or phase of a medical procedure is being displayed on a display 116 presently.

Example system 100 can be utilized for an application (e.g., provided via interfaces 152 or 124) allowing users to search with natural language or natural language with video questions through a library of videos. For example, a questions of a search query 144 could include a request for the system to find another surgery in which a gallbladder has the same level of adhesions as in the current video being displayed on the display 116. For example, a question for the search query 144 can ask for a most complicated cholecystectomy performed by a surgeon. In such instance, surgeon data, such as data associated with a surgeon's profile or surgeon's identifier can be associated with surgeon's performance metrics 134 for various medical procedures. Such data can be used to identify video streams 170 and data streams 162 associated with surgeon's performance data 136 or performance metrics 136.

For example, application operated on interface 152 or 124 can provide surgical video summarization. Such an application can create a text summary of a surgical video to allow a surgeon to read through the key points that occur, and potentially provide a template for their surgical summary. For example, an application can be for surgical video recommendation. This application can recommend similar or different videos for a surgeon to follow up with when doing video review to provide more context to the current video that is being displayed on display 116.

Each of the applications associated with the interfaces 152 or 124 can be based on either a single modal or multimodal embedding data store. For single modal-retrieval augmented generation, the repository 160 can include a single data modality stored with an embedding that allows for semantic search. Embeddings (e.g., 176) can be generated using any model designed for that particular data modality, such as ML models 182 trained for processing images or videos, ML models trained for processing various robotic data streams (e.g., sensors, kinematics or events data) or ML models 182 trained for processing language (e.g., NLP models). The embeddings (e.g., 176) can be stored in any vector database, such as a vector database in a data repository 160.

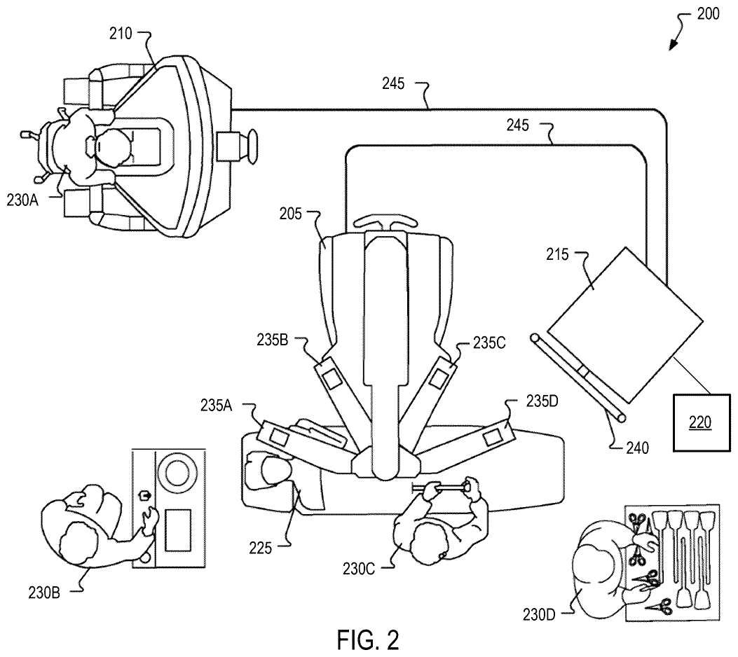

FIG. 2 depicts a surgical system 200, in accordance with some embodiments. The surgical system 200 may be an example of the medical environment 102. The surgical system 200 may include a robotic medical system 205 (e.g., the robotic medical system 120), a user control system 210, and an auxiliary system 215 communicatively coupled one to another. A visualization tool 220 (e.g., the visualization tool 114) may be connected to the auxiliary system 215, which in turn may be connected to the robotic medical system 205. Thus, when the visualization tool 220 is connected to the auxiliary system 215 and this auxiliary system is connected to the robotic medical system 205, the visualization tool may be considered connected to the robotic medical system. In some embodiments, the visualization tool 220 may additionally or alternatively be directly connected to the robotic medical system 205.

The surgical system 200 may be used to perform a computer-assisted medical procedure on a patient 225. In some embodiments, surgical team may include a surgeon 230A and additional medical personnel 230B-230D such as a medical assistant, nurse, and anesthesiologist, and other suitable team members who may assist with the surgical procedure or medical session. The medical session may include the surgical procedure being performed on the patient 225, as well as any pre-operative (e.g., which may include setup of the surgical system 200, including preparation of the patient 225 for the procedure), and post-operative (e.g., which may include clean up or post care of the patient), or other processes during the medical session. Although described in the context of a surgical procedure, the surgical system 200 may be implemented in a non-surgical procedure, or other types of medical procedures or diagnostics that may benefit from the accuracy and convenience of the surgical system.

The robotic medical system 205 can include a plurality of manipulator arms 235A-235D to which a plurality of medical tools (e.g., the medical tool 112) can be coupled or installed. Each medical tool can be any suitable surgical tool (e.g., a tool having tissue-interaction functions), imaging device (e.g., an endoscope, an ultrasound tool, etc.), sensing instrument (e.g., a force-sensing surgical instrument), diagnostic instrument, or other suitable instrument that can be used for a computer-assisted surgical procedure on the patient 225 (e.g., by being at least partially inserted into the patient and manipulated to perform a computer-assisted surgical procedure on the patient). Although the robotic medical system 205 is shown as including four manipulator arms (e.g., the manipulator arms 235A-235D), in other embodiments, the robotic medical system can include greater than or fewer than four manipulator arms. Further, not all manipulator arms can have a medical tool installed thereto at all times of the medical session. Moreover, in some embodiments, a medical tool installed on a manipulator arm can be replaced with another medical tool as suitable.

One or more of the manipulator arms 235A-235D and/or the medical tools attached to manipulator arms can include one or more displacement transducers, orientational sensors, positional sensors, and/or other types of sensors and devices to measure parameters and/or generate kinematics information. One or more components of the surgical system 200 can be configured to use the measured parameters and/or the kinematics information to track (e.g., determine poses of) and/or control the medical tools, as well as anything connected to the medical tools and/or the manipulator arms 235A-235D.

The user control system 210 can be used by the surgeon 230A to control (e.g., move) one or more of the manipulator arms 235A-235D and/or the medical tools connected to the manipulator arms. To facilitate control of the manipulator arms 235A-235D and track progression of the medical session, the user control system 210 can include a display (e.g., the display 116) that can provide the surgeon 230A with imagery (e.g., high-definition 3D imagery) of a surgical site associated with the patient 225 as captured by a medical tool (e.g., the medical tool 112, which can be an endoscope) installed to one of the manipulator arms 235A-235D. The user control system 210 can include a stereo viewer having two or more displays where stereoscopic images of a surgical site associated with the patient 225 and generated by a stereoscopic imaging system can be viewed by the surgeon 230A. In some embodiments, the user control system 210 can also receive images from the auxiliary system 215 and the visualization tool 220.

The surgeon 230A can use the imagery displayed by the user control system 210 to perform one or more procedures with one or more medical tools attached to the manipulator arms 235A-235D. To facilitate control of the manipulator arms 235A-235D and/or the medical tools installed thereto, the user control system 210 can include a set of controls. These controls can be manipulated by the surgeon 230A to control movement of the manipulator arms 235A-235D and/or the medical tools installed thereto. The controls can be configured to detect a wide variety of hand, wrist, and finger movements by the surgeon 230A to allow the surgeon to intuitively perform a procedure on the patient 225 using one or more medical tools installed to the manipulator arms 235A-235D.

The auxiliary system 215 can include one or more computing devices configured to perform processing operations within the surgical system 200. For example, the one or more computing devices can control and/or coordinate operations performed by various other components (e.g., the robotic medical system 205, the user control system 210) of the surgical system 200. A computing device included in the user control system 210 can transmit instructions to the robotic medical system 205 by way of the one or more computing devices of the auxiliary system 215. The auxiliary system 215 can receive and process image data representative of imagery captured by one or more imaging devices (e.g., medical tools) attached to the robotic medical system 205, as well as other data stream sources received from the visualization tool. For example, one or more image capture devices (e.g., data capture devices 110) can be located within the medical environment of the surgical system 200. These image capture devices can capture images from various viewpoints within the surgical system 200. These images (e.g., video streams) can be transmitted to the visualization tool 220, which can then passthrough those images to the auxiliary system 215 as a single combined data stream. The auxiliary system 215 can then transmit the single video stream (including any data stream received from the medical tool(s) of the robotic medical system 205) to present on a display (e.g., the display 116) of the user control system 210.

In some embodiments, the auxiliary system 215 can be configured to present visual content (e.g., the single combined data stream) to other team members (e.g., the medical personnel 230B-230D) who might not have access to the user control system 210. Thus, the auxiliary system 215 can include a display 240 configured to display one or more user interfaces, such as images of the surgical site, information associated with the patient 225 and/or the surgical procedure, and/or any other visual content (e.g., the single combined data stream). In some embodiments, display 240 can be a touchscreen display and/or include other features to allow the medical personnel 230A-230D to interact with the auxiliary system 215.

The robotic medical system 205, the user control system 210, and the auxiliary system 215 can be communicatively coupled one to another in any suitable manner. For example, in some embodiments, the robotic medical system 205, the user control system 210, and the auxiliary system 215 can be communicatively coupled by way of control lines 245, which can represent any wired or wireless communication link that can serve a particular implementation. Thus, the robotic medical system 205, the user control system 210, and the auxiliary system 215 can each include one or more wired or wireless communication interfaces, such as one or more local area network interfaces, Wi-Fi network interfaces, cellular interfaces, etc. It is to be understood that the surgical system 200 can include other or additional components or elements that can be needed or considered desirable to have for the medical session for which the surgical system is being used.

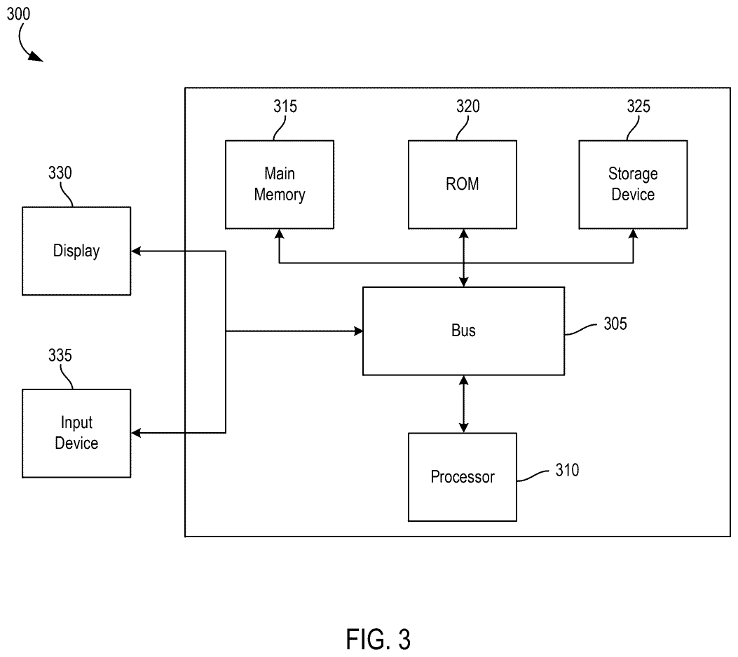

FIG. 3 depicts an example block diagram of an example computer system 300 is shown, in accordance with some embodiments. The computer system 300 can be any computing device used herein and can include or be used to implement a data processing system or its components. The computer system 300 includes at least one bus 305 or other communication component or interface for communicating information between various elements of the computer system. The computer system further includes at least one processor 310 or processing circuit coupled to the bus 305 for processing information. The computer system 300 also includes at least one main memory 315, such as a random-access memory (RAM) or other dynamic storage device, coupled to the bus 305 for storing information, and instructions to be executed by the processor 310. The main memory 315 can be used for storing information during execution of instructions by the processor 310. The computer system 300 can further include at least one read only memory (ROM) 320 or other static storage device coupled to the bus 305 for storing static information and instructions for the processor 310. A storage device 325, such as a solid-state device, magnetic disk or optical disk, can be coupled to the bus 305 to persistently store information and instructions.

The computer system 300 can be coupled via the bus 305 to a display 330, such as a liquid crystal display, or active-matrix display, for displaying information. An input device 335, such as a keyboard or voice interface can be coupled to the bus 305 for communicating information and commands to the processor 310. The input device 335 can include a touch screen display (e.g., the display 330). The input device 335 can also include a cursor control, such as a mouse, a trackball, or cursor direction keys, for communicating direction information and command selections to the processor 310 and for controlling cursor movement on the display 330.

The processes, systems and methods described herein can be implemented by the computer system 300 in response to the processor 310 executing an arrangement of instructions contained in the main memory 315. Such instructions can be read into the main memory 315 from another computer-readable medium, such as the storage device 325. Execution of the arrangement of instructions contained in the main memory 315 can cause the processor 310 or the computer system 300 as a whole to perform the illustrative functionalities or processes described herein. One or more processors in a multi-processing arrangement can also be employed to execute the instructions contained in the main memory 315. Hard-wired circuitry can be used in place of or in combination with software instructions together with the systems and methods described herein. Systems and methods described herein are not limited to any specific combination of hardware circuitry and software.

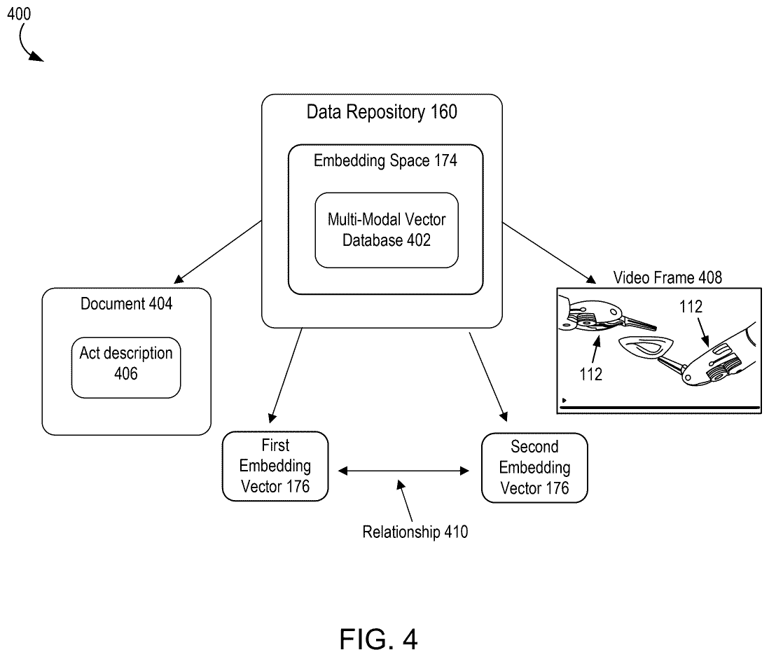

FIG. 4 illustrates an example configuration 400 in which an embedding space implemented with a multi-modal vector database provides relationships between different modes of data. A multi-modal vector database can refer to or include, for example, a data structure that stores embedding vectors for various types of data, such as video, text, sensor, and kinematics data, within a unified embedding space. The multi-modal vector database can allow for efficient semantic search and retrieval across different data modalities by capturing relationships and similarities between diverse data types.

The example configuration 400 can include a data repository 160 storing an embedding space 174 that can include a multi-modal vector database 402. The multi-modal vector database 402 can include various embedding vectors 176 corresponding to various modes of data. The multi-modal vector database 402 (e.g., the embedding space 174) can include and store relationships 410 between the embedding vectors 176 of the respective data modes.

For example, a document 404 can include any medical procedure document that can include an act description 406. The act description 406 can include any textual description of an act. For instance, the act description 406 can include one or more words, phrases or sentences describing a particular medical task within a medical phase of a surgical procedure. The act description 406 can state, for example, that a surgeon is conducting a robotic prostatectomy using a particular medical instrument 112. The act description 406 of the document 404 modality can correspond to, or be described by, a first embedding vector 176. The first embedding vector 176 can include a series of numerical values uniquely describing the contextual meaning or features of the act description 406 within the document 404.

Within the multi-modal vector database 402, a second embedding vector 176 can correspond to, or describe a video frame 408 or a video clip 408 that depicts or corresponds to the act description 406. For instance, the second embedding vector 176 can include a second series of numerical values corresponding to the video frame 408 of the robotic prostatectomy conducted using the particular medical instrument 112 described in the act description 406.

To allow for the multi-modal searching and linkage, the multi-modal vector database 402 can include a relationship 410 linking the first embedding 176 and the second embedding 176. The relationship 410 can include, for example an indicator, a value, a string of characters or a vector with a series of values indicative of the relationship and the linkage between the first embedding 176 and the second embedding. For example, when an embedding vector of a search query matches (e.g., via a search engine 148 of a search query function 142) the act description 406 of the document 404, search query function 142 can search for and identify the relationship 410 associated with the first embedding 176. Based on the relationship 410 with the second embedding 176, the search query function 142 can identify the specific video frame 408 (e.g., or a video clip) corresponding to the search query 144 (e.g., via the first embedding vector 176, the second embedding vector 176 and the relationship 410).

For example, for surgical data, the multi-modal vector database 402 for videos, images, and language can be created using the data streams 162 and video streams 170 that are present in a surgical recording. Videos can be separated into clips and embedded through a video clip level ML model 182. Images can be extracted from video and embedded through an image level ML model 182. Language descriptions can be annotated manually for different video or images, or can be generated automatically, such as from a combination of event data (e.g. stapler fires, tool installations), OPIs, and semantics from kinematics/event streams (e.g. high/low speed, smoothness, jerk, acceleration or force) and external annotations (e.g. phase/step/organ presence).

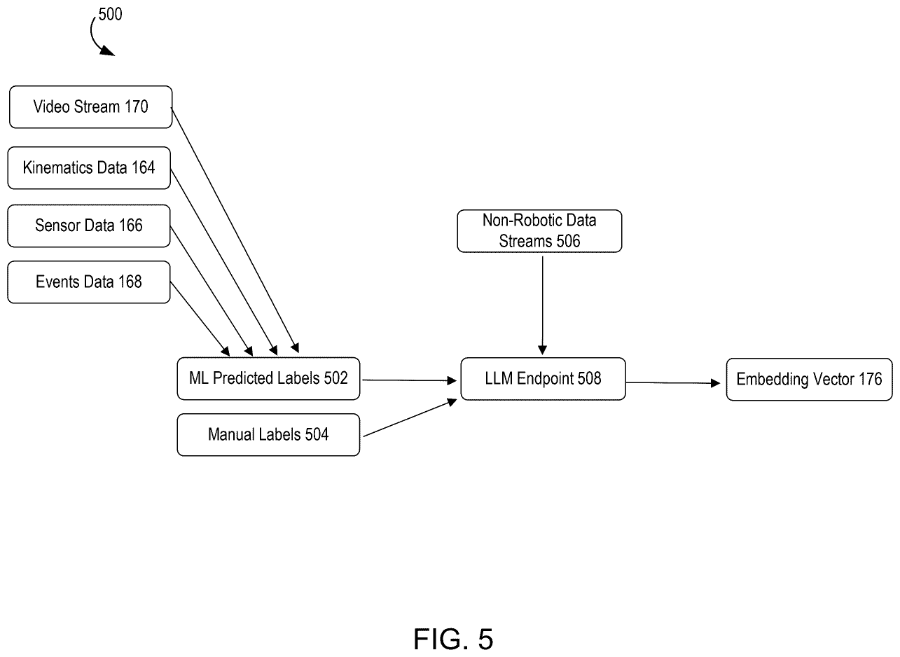

FIG. 5 illustrates an example configuration 500 for generating embedding vectors 176 from data streams of the robotic medical system 120. The example configuration 500 can include video stream 170 or streams of kinematics data 164, sensor data 166 or events data 168 that can have their data labeled using ML techniques, such as ML models 182 trained to predict labels for the portions of these data. The example configuration 500 can include manual labels 504 that can be manually added to the system. The LLM endpoint 508 can receive the ML predicted labels 502 and the manual predicted labels 504 as input. The LLM endpoint 508 can receive non-robotic data streams 506. The non-robotic data streams can include, for example, electronic health records (HER), ultrasound data, magnetic resonance imaging (MRI) data, X-ray images or any other data external to the RMS 120. The LLM endpoint 502 can include one or more ML models 182 trained to process these data according to the ML predicted labels 502 or manual labels 504 and generate embedding vectors 176 for the various portions of the streams of data.

Each of the labels of the various data stream modes (e.g., sensor, kinematics, events and non-robotic data stream 506) be aligned with the relevant portions of the video data stream 170 via timestamps within a surgical procedure. The relationships 410 (e.g., the links) in the multi-modal vector database 402 can be generated automatically for each of these correlated or timestamp linked components to create a large dataset that can include the relationships 410 the various pieces of data.

These features can be used with an application (on interfaces 152 or 124) operating as a surgical data science librarian. In such an application, the process and interaction with such databases can include the database having a set of papers relating to surgical data science that are embedded in a single-modality vector database. The multi-modal vector database 402 can include text vector embeddings 176 with the surgical data to allow searching a specific case/research studies together. The query format can include explanations of different components of surgical data science that are present in either the video review frontend (e.g. “Explain this OPI to me”) or related to research a user is doing on their own data (e.g. “How does this finding compare to the existing literature?”). In response to such search queries 144, the data processing system 130 can respond with a generated answer to the question, along with a set of citations pulled from the multi-modal vector database 402. If the database 402 is connected or has relationship 410 with text embeddings from individual surgeries, the librarian application can pull examples from a specific surgery to support claims in the literature, if available.

These features can be used with an application (on interfaces 152 or 124) for surgical video question and answer. In such an application, a multi-modal vector database 402 can have a set of images and video clips whose embeddings 176 are paired (e.g., have relationship 410) with text vector embeddings 176. In such applications, the search queries can include text, although can involve text and video (e.g., “Take me to the sections where I fire a stapler” or “Describe what is happening at this moment”). In such instances, the data processing system 130 can respond in two ways depending on the presence of labels. For instance, when involving stapler fire, if the labels are present then a text-based semantic search for stapler fires on a database 402 can be performed for annotations/data from this procedure. The data processing system 130 can find the descriptions that have stapler mentioned and return those sections of video with the response 146. When the labels are not present, the system can perform text-based semantic search for stapler fires on a database 402 on annotations/data from other procedures. The system can collect image or clip embeddings 176 where stapler fires are present and use those embeddings 176 to search unsupervised video embeddings from the current procedure to find high likelihood stapler events. If the search query states, “Describe what is happening” and the labels are present, then the system can grab text sections from this timestamp and summarize with LLMs. If the labels are not present, then the similarity search can be performed for video clips or images that are most similar in the embedding database, gather text descriptions of those sections, and summarize the most likely descriptions.

When dealing with a surgical video library search application, the multi-modal vector database 402 can include a set of images and video clips that are paired with a text embedding. In this application, longer video clips (e.g., portions of the video stream 170) can be used than the question and answer application. The search query 144 can have a format referring to multiple videos, such as “find me a complicated cholecystectomy,” or “find me more procedures like the one being displayed now”. Depending on the query, the system can search for text embeddings 176 that match the query and return the video, or video embeddings that match. The LLM agent determine the use for each of these.

When dealing with an application for surgical video summarization, the multi-modal video database 410 can have a set of images and video clips that are paired with a text embeddings 176. The query format can be standardized, to state for example “summarize this procedure”. The system can take a hierarchical approach. First, short clips can be used to search the database for similar videos and label those short clips with text. Then, nearby clips can be aggregated and summarized again using an LLM to repeatedly extract key interesting information, until finally all clips are summarized together to create a procedure summary.

When dealing with surgical video recommendation application, a multi-modal vector database 402 ca be used in which a set of images and video clips can be paired with a text embedding. The search queries 144 can be standardized to search for more similar or different videos. The system response to queries can include both language embeddings and video embeddings which can be used to create a summary score for the similarity of two sessions. Recommendations can then be made based on this summary distance. Clinically relevant recommendations can also be made leveraging the text annotations to limit the recommendation search.

The technical solutions can utilize relationships 410 created between data modalities created by linking multiple surgical data streams based on timestamps at which the multi-modal data were generated within a surgical procedure. This technique can be effective, for instance when data streams are “dense” within a single surgery as there are lots of links that can be used to search across different data modalities. The technical solutions can generate relationships 410 (e.g., links) between various modes of data in a variety of ways that can provide different levels of connection and help to solve problems, such as when labels are sparse.

For example, the technical solutions can utilize different methodologies within a single modality, such as distance-based nearest neighbor search. For instance, when a query vector is passed to a system, pairwise distances between that query vector 176 and the vectors 176 of the database 410 can be computed, and the nearest neighbors can be used to return a response to identify the most “similar” vectors. This could be augmented to also find the most “different” vectors, as desired. For example, the technical solutions can utilize a linear model development. For example, distances in an embedding space can correspond to a variety of different “features” of a data point (e.g. organs present, tools present, anatomy state, camera settings can all be ‘summarized’). When specific features are of interest, a small set of example can be provided (positive and negative of a class), and a linear model can be used to separate these two classes specifically. For example, technical solutions can utilize interpolation through generative embedding spaces (synthetic data). For instance, if embedding spaces 174 are generative, interpolation between points in the embedding space 174 can produce sensible (synthetic) data. For example, this can be useful to describe unseen situations, or describe a point that is not directly similar to any existing points in the embedding space.

To provide relationships 410 (e.g., linkages) across different data modalities, the technical solutions can utilize time-based linkages. For instance, time-based linkages can be based on data from a single surgery being linked across multiple data modalities using timestamps of the different data modalities. For instance, function mapping can be used to link (e.g., create relationships 410) for data across data modalities, allowing for search to expand. These functions can be used to compress data (e.g. representation in one modality is similar across multiple data points) or allow for broader search (e.g., representation in one modality maps to a broad spectrum of representations in other data modalities). This technique can create more relationships 410 (e.g., links) between data points, which can be useful when labels are sparse and single time-based links are not dense in the dataset.

For instance, multi-modal embedding spaces (generative/non generative) can be provided where ML models 182 can create embedding spaces 174 that are shared between two or more data modalities allowing search to happen instantly with multiple decoders (one per modality). These spaces can be generative, allowing for interpolation within the embedding space 174 and generation of data in each modality for these synthetic data points. For instance, human in the loop can be used for additional annotations of data. The links between data types can be generated by human in the loop annotations to create denser links between data. For example, ultrasound data could be paired with endoscopic data, and humans could determine whether or not the pairing was logical.

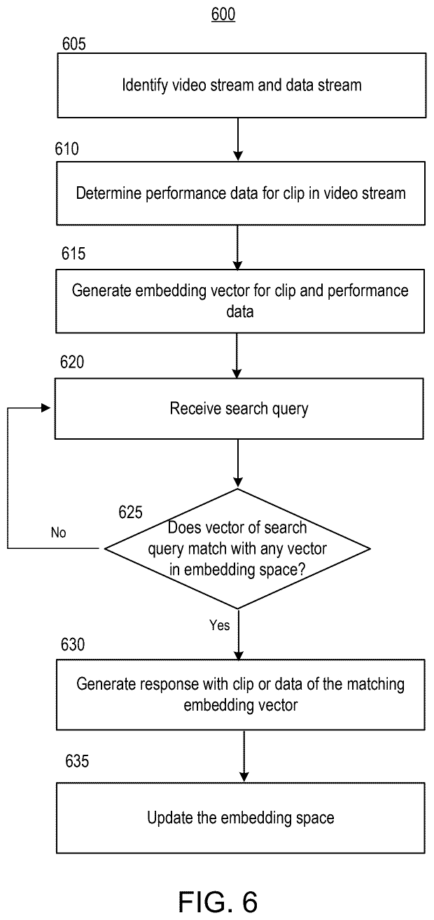

FIG. 6 illustrates an example flow diagram of a method 600 for a multi-modal retriever augmented generation for natural language reactions. The method 600, can be performed by a system having one or more processors (e.g., 310) configured to perform operations of the system 100 by executing computer-readable instructions stored on a memory (e.g., 315). For instance, method 600 can be implemented using a non-transitory computer readable medium storing instructions that, when executed by one or more processors (e.g., 310), cause the one or more processors (e.g., 310) to implement operations or acts of the method. The method 600 can be performed, for example, in accordance with any features or techniques discussed in connection with FIGS. 1-3.

The method 600 can include operations 605-440. At operation 605, the method can identify a video stream and a data stream. At operation 610, the method can determine performance data for a clip in the video stream. At operation 615, the method can generate an embedding vector for the video clip and the performance data. At operation 620, the method can receive a search query. At operation 625, the method determine if the embedding vector of the search query match any of the embedding vectors in the embedding space. At 630, responsive to the vector of the search query matching an embedding vector at the embedding space, the method can generate a response with the video clip or data associated with the matching embedding vector. At 635, the method can update the embedding space. At 640, the method can provide the response for display.

At operation 605, the method can identify a video stream and a data stream. The method can include one or more processors that are coupled with memory and configured to identify one or more modalities (e.g., types) of data associated with a medical procedure. The one or more modalities of data can include data streams generated by a robotic medical system (e.g., robotic system controllers or sensors) or by components of a medical environment (e.g., data capture devices or sensors deployed in the operating room). The one or more modalities of data can include data streams, or portions of data streams, corresponding to video data, such as video clip or a series of images or video frames from one or more cameras. The one or more modalities of data can include streams or portions of streams of sensor data, kinematics data or events data from a robotic medical system. The one or more modalities of data can include data or streams of data, or portions of streams, that are not generated by the robotic medical system or its components, such as X-ray imaging data, magnetic resonance imaging (MRI) data, electronic health records (EHR) data, patient health data (e.g., data on patient's prior conditions), surgeon data (e.g., data on surgeon's prior performance, performance metrics and records of surgical procedures of the surgeon) or any other medical or health related data.

For example, the method can include the one or more processors identifying, for a medical procedure performed via a robotic medical system, a video stream (e.g., a sequence of video frames from an endoscopic device or another medical instrument) and a plurality of data streams related to the medical procedure (e.g., sensor, kinematics or events data). For example, the one or more processors can receive, from the robotic medical system, the plurality of data streams comprising at least one of a kinematics data stream, an event stream, or a non-robotic data stream.

The method can include the one or more processors identifying data based on the data currently being displayed on the displayed. For example, the method can include displaying, via a graphical user interface, the video clip of the medical procedure. For instance, a graphical user interface of an application can display the video of the medical procedure on a display monitor.

During the displaying of the video clip, the method can include receiving, via the graphical user interface, a query related to the medical procedure. For example, the query can be a search query that that user can generate referencing the video currently being displayed or referencing a particular movement or a particular task performed in the video, a particular phase of a medical procedure in the video or a particular medical procedure. The query can be a search query seeking information or data related to a portion or a detail of the video being displayed. The method can include referencing the video or the portion of the video.

At operation 610, the method can determine performance data for a clip in the video stream. The method can include the one or more processors determining, using one or more models trained with machine learning, based on the plurality of data streams, performance data for a clip of the video stream. The performance data can include performance metrics associated with the duration of the video clip. The performance data can include objective performance metrics (OPIs) of a surgeon performance during the duration of the video clip (e.g., portion of video stream). The performance data can be determined based on the video stream or any of the data streams (e.g., sensor, kinematics, events or non-robotic data). The one or more ML models used to determine the performance data can utilize any one or more of a portion of video stream or data stream (e.g., sequence of sensor, events or kinematics data or readings) for the determination. For instance, the video data or the data stream portions can be input into the ML model trained to determine the performance data (e.g., any of the OPIs) based on the input.

Performance data can be generated using sensor, kinematics, or events data, and can include metrics corresponding to performance with respect to particular tasks or actions. For instance, performance data can include a total duration of specific actions, tasks or phases of the procedure, measured in minutes or seconds. Performance data can include a range (e.g., maximum and minimum) or average amount of force applied by surgical instruments during a particular action. Performance data can include the number of times a medical instrument is engaged or disengaged, various kinematic trajectories for various movement patterns of surgical instruments, speed or direction of movements or any other data. Performance data can include timestamps or time durations for particular events (e.g., actions, tasks or phases) identifying duration, start or end of an occurrence, allowing to identify concurrent video, sensor or kinematics data to relate in the embedding space. Performance data can include or correspond to amount of medicine administered, amount of blood detected in a particular task or procedure, a number of clutch counts performed on hand controllers or endoscopes, or patient response data (e.g., vital signals of the patient, including heart rate, oxygen levels, blood pressure and similar).

The one or more ML models can include a generative artificial intelligence model (Gen AI model) and the one or more processors can be configured to generate, using the generative artificial intelligence model, the performance data based on the plurality of data streams. The performance data can include a text-based description (e.g., text description) of the video clip which can be generated from the plurality of data streams. For instance, the text description can be generated by the Gen AI model based on the data streams input into the Gen AI model.

The method can include generating, using the one or more ML models, a plurality of performance metrics. The performance metrics can be generated based on the plurality of data streams input into the one or more ML models which can be trained to generate or determine the performance metrics based on the data streams or video stream input into the one or more ML models. The method can include generating, using generative artificial intelligence model, the performance data based on the plurality of performance metrics.

At operation 615, the method can generate an embedding vector for the video clip and the performance data. The method can include transforming the performance data and the clip to an embedding vector for an embedding space stored in a data repository. The transformation of the performance data can include the embedding vector generator constructing, generating or providing an embedding vector for the video clip. The embedding vector generator can construct, create or generate embedding vectors for any portions of the data streams, including streams of data from sensors, kinematics and events of the robotic medical system. The embedding vector generator can construct, create or generate embedding vectors for any non-robotic data sources, such as including data from X-ray machines, MRIs, EHR data, surgeon data or patient data.

The method can include the embedding vector generator or the embedding space function establishing, determining or creating relationships or linkages between different vectors pertaining to the same or different modalities of data. For example, the embedding space function can generate a vector database comprising relationships between embeddings of video clips and embeddings of data stream portions (e.g., sensor data, kinematics data or events data). The embedding space function can generate relationships between the vectors of the same or different data modalities based on the timestamps of each of the pieces of data (e.g., video clips and concurrent sensor or kinematics data). The method can utilize timestamps in the metadata or vector of each of the pieces of video or data streams to relate or define relationship between various vectors and their corresponding data.

At operation 620, the method can receive a search query. The method can include a user interface of an application executed by a user of a client device receiving an input from the user. The input can include a search query which can include a textual input, such as a string of characters, including one or more words, phrases or sentences. The search query can include a natural language query, describing a particular search to perform across one or more modes of data. For instance, the search query can include a text requesting a search of a particular portion of a video along with any corresponding non-video (e.g., data stream) data, including any sensor, kinematics or events data.

The search query can be received via a user interface of a client device, which can include a work station of a medical environment in which the user (e.g., a surgeon) can request the data processing system to search various video recordings and the corresponding data streams of various medical procedures to identify a particular portion of a video clip matching the description of the query. The method can provide a graphical user interface for a search engine. The method can receive, via the graphical user interface, the search query. The method can utilize the search query function to process the search query using one or more search engines, across the embedding space, and identify or generate the response comprising the relevant video portion or its data.

At operation 625, the method determine if the embedding vector of the search query match any of the embedding vectors in the embedding space. The one or more processors can utilize the embedding vector generator to generate an embedding vector for the search query. The search query function can compare the embedding vector of the search query against any number of embedding vectors of the embedding space (e.g., multi-modal vector databased) and identify embedding vectors in the embedding space that semantically most closely match the embedding vector of the search query. To identify the most closely matching vectors of the embedding space, the search query function can utilize the search engine. The search query function can perform semantic similarity functions or comparisons between the search query vectors and various vectors of the embedding space.

The one or more processors can implement the search query function that can utilize any similarity functions or techniques to identify or select the most closely matching video clips or their corresponding data stream portions. For instance, the one or more processors can execute the search query using a distance-based nearest neighbor search between the vector of the search query and the vectors in the embedding space. The one or more processors can identify the closest distance-based nearest neighbor and identify the video or stream data of that vector as the matching vector. For instance, the one or more processors can execute the search query using a linear model. The search query function can utilize the embedding space function to identify any vectors that are related to (e.g., in relationship with) the identified video clip. For instance, the embedding space function can identify a portion of data stream that has a relationship or linkage with the video clip. The search query function can identify or select the portion of the data stream related to the video clip to provide with the response, along with the selected matching video clip. The one or more processors can execute the search query via interpolation through a generative embedding space to identify the search result. The search result can include synthetic data.

If the search query function does not find any entries using one technique, the search query function can resort to other techniques. For instance, if the search query function does not find a video clip of a particular action or task implemented in one medical procedure, it can identify a video clip of the same action or task implemented in another medical procedure. For example, if the search query function does not identify a vector of a video clip or data matching the search query embedding vector for a given medical procedure requested, it can generate a follow up question back to the user interface if a video clip or data of a different medical procedure is acceptable. In response to an approval from the user interface, the search query function can proceed and provide the video clip of the related or approved different medical procedure. If, however, the search query function identifies no matches, the search query function can trigger a response to the user interface that no match is found and go back to act 620 to wait for the next search query.

At 630, responsive to the vector of the search query matching an embedding vector at the embedding space, the method can generate a response with the video clip or data associated with the matching embedding vector. The one or more processors can provide a response to the search query comprising the video clip or a link to the video clip identified or selected at act 625. The one or more processors can provide the response comprising a portion of the data stream (e.g., a sequence of sensor readings, kinematics data or events data) that is related to the video clip. Responsive to the search query received via a graphical user interface (e.g., at a client device), the one or more processors can select, based on execution of the search query on the embedding space, the search result that corresponds to the medical procedure referenced in the search query.

For example, the one or more processors can receive, during the display of the clip, via the graphical user interface, a query related to the medical procedure. The query can reference or correspond to the video clip being displayed during the time when the search query is being generated. The one or more processors can execute the query on the embedding space to select the performance data associated with the video clip referenced in the query and then provide a response to the query based at least in part on the performance data. For instance, the method can include selecting, based on execution of the search query on the embedding space, a search result corresponding to the medical procedure referenced in the search query.

The one or more processors can utilize one or more ML models to use the matching or related data stream portions to generate text-based output referencing the related video clip (e.g., video clip that is determined to be in relationship with the given data stream portions). The text-based output can describe the data stream data, such as sensor data, kinematics data or events data to be displayed together with the video clip. The one or more ML models can generate textual summary of the data stream portions in reference to the action, task or phase depicted in the video clip.

At 635, the method can update the embedding space. The method can include the one or more processors updating the embedding space to provide access to at least a portion of the video stream of the medical procedure. The one or more processors can update the embedding space in response to the search query executed on, or using, the embedding space. Embedding space can be updated to include, into the embedding space, an embedding vector of the video clip or a relationship between the embedding vector and one or more portions of one or more data streams (e.g., sensor, kinematics or events data).

The one or more processors can update the embedding space with a plurality of embedding vectors constructed for a plurality of clips of the video stream. The one or more processors can aggregate performance data for at least two of the plurality of clips. The one or more processors can generate an aggregated embedding vector for the aggregated performance data. The one or more processors can update the embedding space with the aggregated embedding vector. The one or more processors can update the embedding space with a plurality of embedding vectors constructed for a plurality of clips of a plurality of video streams of a plurality of medical procedures.

At 640, the method can provide the response for display. The method can include providing for display the response referencing or including the video clip and the related portions of data stream. For instance, the interface of the data processing system can transmit the response, via the network, to the user interface of a client device from which the search requested was generated. The response can include link to, or reference, the video clip identified as matching at act 625 and any portions of data stream (e.g., sensor data, kinematics data or events data) determined to have relationship or linkage with the matching video clip.

The video clip can be displayed to the user via the graphical user interface. The user interface can display, via the user interface, the portions of the data streams related to the video clip, such as portions of the data stream that was generated during the time period during which the video clip was generated. The user interface can display the textual output generated by the ML models from the data stream portions determined to be in relationship with the video clip. The user interface can display a summary of the textual output in context with, or describing details related to the action or task displayed in the video clip. For instance, the user interface can display, along with the video clip, a text-based description of the force applied by a medical instrument (e.g., sensor data), a time or type of engagement of a medical instrument (e.g., event data), or direction of swipe or movement of a medical instrument (e.g., kinematics data). Such text-based descriptions can be displayed alongside the video clip, or can be narrated (e.g., provided via audio output) with the video clip.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

With respect to the use of plural or singular terms herein, those having skill in the art can translate from the plural to the singular or from the singular to the plural as is appropriate to the context or application. The various singular/plural permutations can be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description can illustrate a specific order of method steps, the order of such steps can differ from what is depicted and described, unless specified differently above. Also, two or more steps can be performed concurrently or with partial concurrence, unless specified differently above. Such variation can depend, for example, on the software and hardware systems chosen and on designer choice. All such variations are within the scope of the disclosure. Likewise, software implementations of the described methods can be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or can be acquired from practice of the disclosed implementations. It is intended that the scope of the invention be defined by the claims appended hereto and their equivalents.

What is claimed is:

1. A system, comprising:
one or more processors, coupled with memory, to:
identify, for a medical procedure performed via a robotic medical system, a video stream and a plurality of data streams related to the medical procedure;
determine, based on the plurality of data streams, performance data for a clip of the video stream;
transform the performance data and the clip to an embedding vector for an embedding space stored in a data repository; and
update the embedding space to provide, in response to a search query related to performance of the medical procedure, access to at least a portion of the video stream of the medical procedure.

2. The system of claim 1, wherein the one or more processors are further configured to:
receive, from the robotic medical system, the plurality of data streams comprising at least one of a kinematics data stream, an event stream, or a non-robotic data stream.

3. The system of claim 1, wherein the one or more processors are further configured to:
generate, using a generative artificial intelligence model, the performance data based on the plurality of data streams.

4. The system of claim 3, wherein the performance data includes a text-based description of the clip generated from the plurality of data streams.

5. The system of claim 1, wherein the one or more processors are further configured to:
generate, using one or more models trained with machine learning, a plurality of performance metrics based on the plurality of data streams; and
generate, using generative artificial intelligence, the performance data based on the plurality of performance metrics.

6. The system of claim 1, wherein the one or more processors are further configured to:
provide a graphical user interface for a search engine;
receive, via the graphical user interface, the search query;
select, based on execution of the search query on the embedding space, a search result corresponding to the medical procedure; and
provide the search result for display via the graphical user interface.

7. The system of claim 6, wherein the one or more processors are further configured to:
execute the search query using a distance-based nearest neighbor search.

8. The system of claim 6, wherein the one or more processors are further configured to:
execute the search query using a linear model.

9. The system of claim 6, wherein the one or more processors are further configured to:
execute the search query via interpolation through a generative embedding space to identify the search result, wherein the search result comprises synthetic data.

10. The system of claim 1, wherein the one or more processors are further configured to:
display, via a graphical user interface, the clip of the medical procedure;
receive, during the display of the clip, via the graphical user interface, a query related to the medical procedure;
execute the query on the embedding space to select the performance data associated with the clip; and
provide a response to the query based at least in part on the performance data.

11. The system of claim 1, wherein the one or more processors are further configured to:
update the embedding space with a plurality of embedding vectors constructed for a plurality of clips of the video stream.

12. The system of claim 11, wherein the one or more processors are further configured to:
aggregate performance data for at least two of the plurality of clips;
generate an aggregated embedding vector for the aggregated performance data; and
update the embedding space with the aggregated embedding vector.

13. The system of claim 1, wherein the one or more processors are further configured to:
update the embedding space with a plurality of embedding vectors constructed for a plurality of clips of a plurality of video streams of a plurality of medical procedures.

14. A method, comprising:
identifying, by one or more processors coupled with memory, for a medical procedure performed via a robotic medical system, a video stream and a plurality of data streams related to the medical procedure;
determining, by the one or more processors, based on the plurality of data streams, performance data for a clip of the video stream;
transforming, by the one or more processors, the performance data and the clip to an embedding vector for an embedding space stored in a data repository; and
updating, by the one or more processors, the embedding space to provide, in response to a search query executed on the embedding space, access to at least a portion of the video stream of the medical procedure.

15. The method of claim 14, comprising:
receiving, by the one or more processors, from the robotic medical system, the plurality of data streams comprising at least one of a kinematics data stream, an event stream, or a non-robotic data stream.

16. The method of claims 14, comprising:
generating, by the one or more processors, using a generative artificial intelligence model, the performance data based on the plurality of data streams, wherein the performance data includes a text-based description of the clip generated from the plurality of data streams.

17. The method of claim 14, comprising:
generating, by the one or more processors, using one or more models trained with machine learning, a plurality of performance metrics based on the plurality of data streams; and
generating, by the one or more processors, using generative artificial intelligence, the performance data based on the plurality of performance metrics.

18. The method of claim 14, comprising:
displaying, by the one or more processors, via a graphical user interface, the clip of the medical procedure;
receiving, by the one or more processors, during the display of the clip, via the graphical user interface, a query related to the medical procedure;
executing, by the one or more processors, the query on the embedding space to select the performance data associated with the clip; and
providing, by the one or more processors, a response to the query based at least in part on the performance data.

19. A non-transitory computer-readable medium storing processor executable instructions that, when executed by one or more processors, cause the one or more processors to:
identify, for a medical procedure performed via a robotic medical system, a video stream and a plurality of data streams related to the medical procedure;
determine, based on the plurality of data streams, performance data for a clip of the video stream;
transform the performance data and the clip to an embedding vector for an embedding space stored in a data repository; and
update the embedding space to provide, in response to a search query executed on the embedding space, access to at least a portion of the video stream of the medical procedure.

20. The non-transitory computer-readable medium of claim 19, wherein the instructions further include instructions to:
generate, using a generative artificial intelligence model, the performance data based on the plurality of data streams.
